(19) United States
(12) Patent Application Publication
(10) Pub. No.: US 2025/0120725 A1
(43) Pub. Date: Apr. 17, 2025

(54) CARTRIDGE SYSTEM

(71) Applicant: OLYMPUS MEDICAL SYSTEMS CORP., Tokyo (JP)

(72) Inventors: Yutaka YANUMA, Hachioji-shi (JP); Shogo Shindo, Hachioji-shi (JP); Jun Shirai, Hachioji-shi (JP); Ryoma Kita, Hachioji-shi (JP); Kensuke Uesaka, Hachioji-shi (JP); Yuya Hidaka, Hachioji-shi (JP); Hiroshi Minami, Hachioji-shi (JP)

(21) Appl. No.: 18/917,969

(22) Filed: Oct. 16, 2024

Related U.S. Application Data
(60) Provisional application No. 63/590,939, filed on Oct. 17, 2023.

(51) Int. Cl. A61B 17/128

(52) U.S. Cl. CPC A61B 17/1285

(57) ABSTRACT

A cartridge system includes a main body portion having a plurality of clip storage areas. Each of the clip storage areas stores a clip, and each of the clip storage areas is provided with an opening through which an applicator can be inserted and a lid for independently opening and closing the opening.

### CROSS-REFERENCE TO RELATED APPLICATION

This application is based on and claims priority under 35 U.S.C. § 119 to U.S. Provisional Application No. 63/590,939, filed Oct. 17, 2023, the entire contents of which are incorporated herein by reference.

### BACKGROUND

### Technical Field

This disclosure relates to a cartridge system that houses an endoscopic clip unit.

### Description of Related Art

In endoscopic treatment, a clip unit is used that can ligate the resection area after treatment to stop bleeding. The clip unit includes a clip that clamps the resection area, and a pressure tube that houses the clip and locks it in a closed state. The clip unit is introduced to the treatment position by an applicator (introduction device) that can be inserted through the channel of the endoscope.

The clip unit is loaded into the clip introduction device using the cartridge system. PCT International Publication No. WO 03/053256 (Patent Document 1) and PCT International Publication No. WO 04/082488 (Patent Document 2) describe a cartridge system in which multiple clip units are stored.

However, in the conventional cartridge systems described in Patent Documents 1 and 2, etc., it is not considered that unused clip units that have not been removed from the cartridge system in a case will be used in a different case.

### SUMMARY

The present disclosure provides a cartridge system that allows unused clip units from a plurality of clip units to be used in different cases.

The present invention proposes the following.

A cartridge system according to a first aspect of the present disclosure includes a main body portion having a plurality of clip storage areas. Each of the clip storage areas stores a clip, and each of the clip storage areas is provided with an opening through which an applicator can be inserted and a lid for independently opening and closing the opening.

The cartridge system of the present disclosure allows unused clip units from among a plurality of clip units to be used in different cases.

### BRIEF DESCRIPTION OF THE DRAWINGS

FIG. 1 is a perspective view showing a clip system according to a first embodiment.

FIG. 2 is a diagram showing a clip storage area of a cartridge system in the clip system.

FIG. 3 is a perspective view of a clip introduction device.

FIG. 4 is a perspective view of a clip unit.

FIG. 5 is a perspective view of the clip unit.

FIG. 6 is a longitudinal cross-sectional view of a pressing member of the clip unit.

FIG. 7 is a perspective view of a pressing tube of the pressing member.

FIG. 8 is a perspective view of the pressing tube as viewed from the proximal end side.

FIG. 9 is a perspective view of a connecting member of the clip unit.

FIG. 10 is a diagram showing a method of loading the clip unit into the clip introduction device using the cartridge system.

FIG. 11 is a diagram showing a method of loading the clip unit into the clip introduction device using the cartridge system.

FIG. 12 is a diagram showing a method of loading the clip unit into the clip introduction device using the cartridge system.

FIG. 13 is a diagram showing a method of loading the clip unit into the clip introduction device using the cartridge system.

FIG. 14 is a diagram showing a method of loading the clip unit into the clip introducer using the cartridge system.

FIG. 15 is a diagram showing the clip unit introduced into a body.

FIG. 16 is a diagram showing the clip unit with a pair of arms closed.

FIG. 17 is a diagram showing the clip unit with the clip locked.

FIG. 18 is a diagram showing the clip unit with the clip separated.

FIG. 19 is a diagram showing an example of an implementation form of the cartridge system.

FIG. 20 is a diagram showing another example of an implementation form of the cartridge system.

FIG. 21 is a diagram showing another example of an implementation form of the cartridge system.

FIG. 22 is a diagram showing a modified example of the cartridge system.

FIG. 23 is a diagram showing a modified example of the seal.

FIG. 24 is a diagram showing a cartridge system according to a second embodiment.

FIG. 25 is a diagram showing a modified main body portion and a modified base of the cartridge system.

FIG. 26 is a diagram showing a modified example of the cartridge system.

FIG. 27 is a diagram showing another aspect of the modified example of the cartridge system.

FIG. 28 is a diagram showing another aspect of the modified example of the cartridge system.

FIG. 29 is a diagram showing another aspect of the modified cartridge system.

FIG. 30 is a diagram showing another aspect of the modified cartridge system.

FIG. 31 is a diagram showing another aspect of the modified cartridge system.

FIG. 32 is a diagram showing a cartridge system according to a third embodiment.

FIG. 33 is a diagram showing a first main body portion arranged in an insertable position.

FIG. 34 is a diagram showing a main body portion with the sheath of the clip introduction device inserted therein.

FIG. 35 is a diagram showing the main body portion with the sheath of the clip introduction device pulled out.

FIG. 36 is a diagram showing a clip introduction device of a clip system according to a fourth embodiment.

FIG. 37 is a diagram showing a cartridge system of the clip system.

FIG. 38 is a diagram showing the sheath of the clip introduction device inserted into a clip storage area.

FIG. 39 is a diagram showing a divided connecting member.

FIG. 40 is a cross-sectional view taken along line X-X shown in FIG. 39.

FIG. 41 is a diagram showing the clip unit after loading.

FIG. 42 is a diagram showing the right main body portion and the left main body portion spaced apart in the width direction.

FIG. 43 is a diagram showing the clip unit removed from the storage case.

### DETAILED DESCRIPTION

### First Embodiment

### [Clip System 1000]

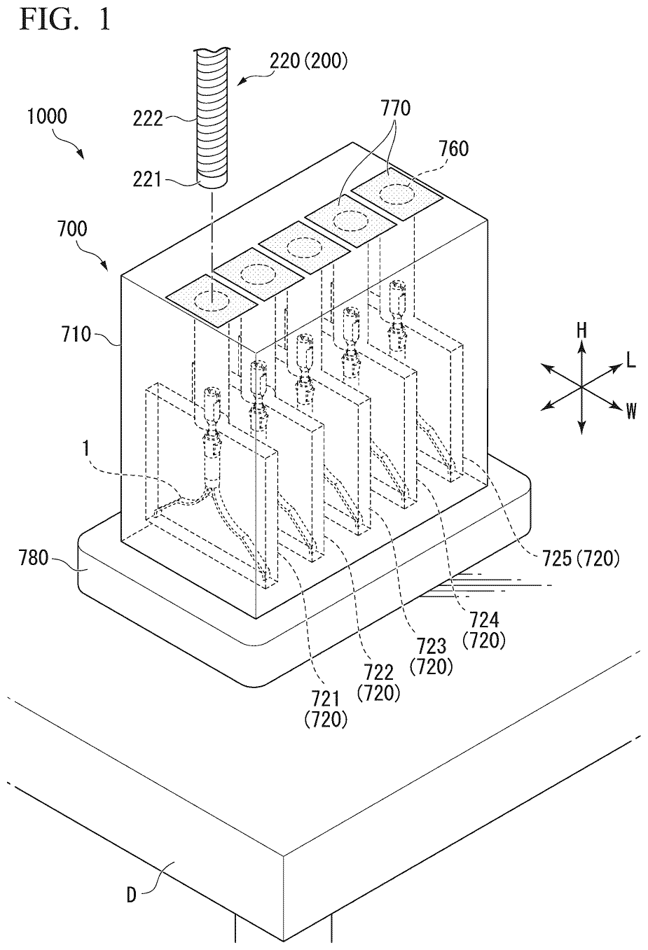

A clip system 1000 according to a first embodiment of the present disclosure will be described with reference to FIGS. 1 to 31. FIG. 1 is a perspective view showing the clip system 1000. The clip system 1000 includes a cartridge system 700 and a clip introduction device (applicator) 200.

### [Cartridge System 700]

The cartridge system 700 loads a clip unit 1 into the clip introduction device 200. The cartridge system 700 stores a plurality of clip units 1 that can be loaded into the clip introduction device 200. The cartridge system 700 may store only one clip unit 1.

The cartridge system 700 can be stood on a mounting surface such as a workbench D as shown in FIG. 1. Specifically, the cartridge system 700 includes a main body portion 710 in which a plurality of clip units 1 are stored, and a base 780 that is disposed on the mounting surface.

In the following description, the direction of gravity in the cartridge system 700 standing on a support surface such as a workbench D is defined as the “height direction (up and down direction) H”, the direction perpendicular to the height direction His defined as the “longitudinal direction L”, and the direction perpendicular to the height direction H and the longitudinal direction L is defined as the width direction W.

The main body portion 710 is formed in a rectangular parallelepiped shape extending in the height direction H, the longitudinal direction L, and the width direction W. The length of the longitudinal direction Lis longer than the length of the width direction W. The shape of the main body portion 710 is not limited to this and may be other shapes.

The main body portion 710 has multiple clip storage areas 720. The main body portion 710 illustrated in FIG. 1 has five clip storage areas 720 (first clip storage area 721, second clip storage area 722, third clip storage area 723, fourth clip storage area 724, and fifth clip storage area 725). The first clip storage area 721, the second clip storage area 722, the third clip storage area 723, the fourth clip storage area 724, and the fifth clip storage area 725 are arranged in this order along the longitudinal direction L. The number and arrangement of the multiple clip storage areas 720 in the main body portion 710 are not limited to this.

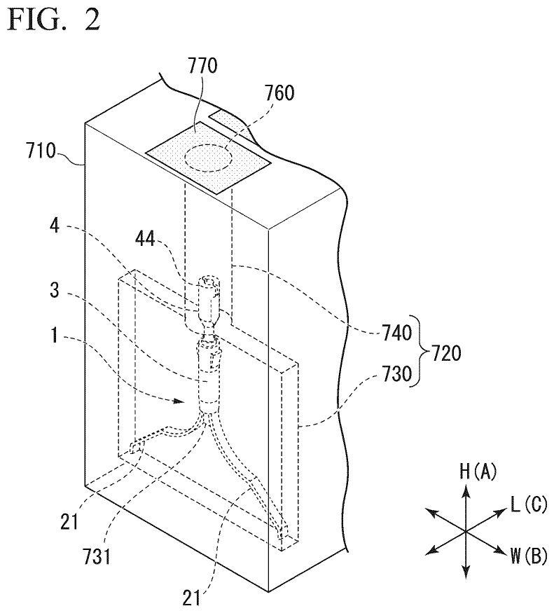

FIG. 2 is a diagram showing the clip storage areas 720.

The clip unit 1 is stored in each of the multiple clip storage areas 720. The clip storage area 720 has a deployment area 730 in which the clip unit 1 is deployed, and a sheath insertion area 740 in which the sheath 220 of the clip introduction device 200 is inserted.

The deployment area 730 is an area in which the clip unit 1 is mainly stored and is a rectangular parallelepiped internal space. The deployment area 730 is provided with a stopper 731 that restricts the clip unit 1 from moving downward in the height direction H.

The sheath insertion area 740 is an area where the sheath 220 of the clip introduction device 200 is inserted and is a cylindrical internal space. The sheath insertion area 740 is connected to the deployment area 730.

Each of the multiple clip storage areas 720 is provided with an opening 760 into which the sheath 220 of the clip introduction device 200 can be inserted, and a lid 770 that closes the opening 760 in an independently releasable manner. The opening 760 is connected to the sheath insertion area 740. When the opening 760 is closed by the lid 770, the clip storage area 720 is maintained in a sterile state.

The lid 770 illustrated in FIG. 2 is a seal that closes the opening 760. The opening 760 is opened when the sheath 220 of the clip introduction device 200 breaks through the lid 770 and enters the sheath insertion area 740. The lid 770 is not limited to a seal that is broken by the sheath 220 but may be a seal that can be peeled off by the user. The seal that can be peeled off by the user is preferably a seal that allows confirmation that the seal, such as a security seal, has been broken, or a seal that cannot be reattached once peeled off. The lid 770 may be a snap-type rubber stopper that can be fitted into the opening 760, or a hard resin cover that covers the opening 760.

The base 780 is a flat plate-shaped member that is disposed on a placement surface such as a workbench D. The base 780 supports the main body portion 710 with the opening 760 positioned at a position where the sheath 220 of the clip introduction device 200 can be inserted. The base 780 illustrated in FIG. 1 supports the main body portion 710 with the opening 760 positioned on the upper side in the height direction H. The main body portion 710 and the base 780 may be formed integrally.

### [Clip Introduction Device 200]

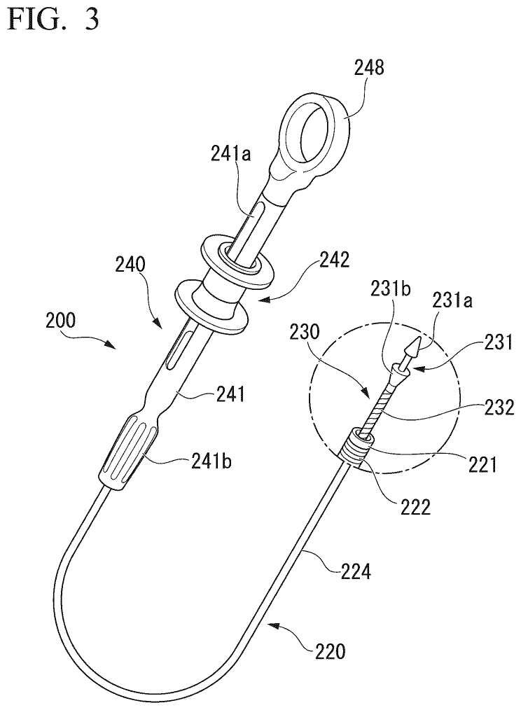

FIG. 3 is a perspective view of the clip introduction device 200.

The clip introduction device (applicator) 200 includes a sheath 220, an operation wire 230, and an operation portion 240. The clip introduction device 200 is inserted, for example, into a treatment tool insertion channel of an endoscope and used in combination with the endoscope. For this reason, the sheath 220 is formed to be sufficiently longer than the length of the treatment tool insertion channel of the endoscope. The sheath 220 is flexible and curves in accordance with the curvature of the insertion section of the endoscope.

The sheath 220 includes a distal chip 221, a distal coil 222, and a proximal coil 224, and is formed into an elongated tube shape. The distal coil 222 is disposed on the distal end side of the sheath 220. The distal chip 221 is disposed at the distal end of the distal coil 222.

The operation wire (power transmission portion) 230 includes an arrowhead hook (connecting portion) 231 connected to the clip unit 1, and a wire 232 that operates the arrowhead hook 231.

The arrowhead hook 231 includes an approximately conical engaging portion 231a that engages with the clip unit 1, and a wire connecting portion 231b provided at the proximal end of the engaging portion 231a. The arrowhead hook 231 is formed of a metal material such as stainless steel.

The wire 232 is inserted through the sheath 220 so as to be advanceable and retractable. The distal end of the wire 232 is fixed to the proximal end of the wire connecting portion 231b by, for example, welding.

The operation portion 240 includes an operation portion main body portion 241, a slider 242, and a thumb ring 248. The operation portion main body portion 241, the slider 242, and the thumb ring 248 are injection molded from, for example, a resin material. The operation portion main body portion 241 has a slit portion 241a and a rotating grip 241b on the distal end side. The slit portion 241a supports the slider 242 so as to be advanceable and retractable.

The slider 242 is attached to the operation portion main body portion 241 so as to be advanceable and retractable in the longitudinal axis direction, and the proximal end of the wire 232 is attached to it. When the slider 242 advances and retracts along the operation portion main body portion 241, the wire 232 advances and retracts relative to the sheath 220, and the arrowhead hook 231 advances and retracts.

The thumb ring 248 is attached to the proximal end of the operation portion main body portion 241 so that it can rotate around the longitudinal axis of the operation portion main body portion 241.

### [Clip Unit 1]

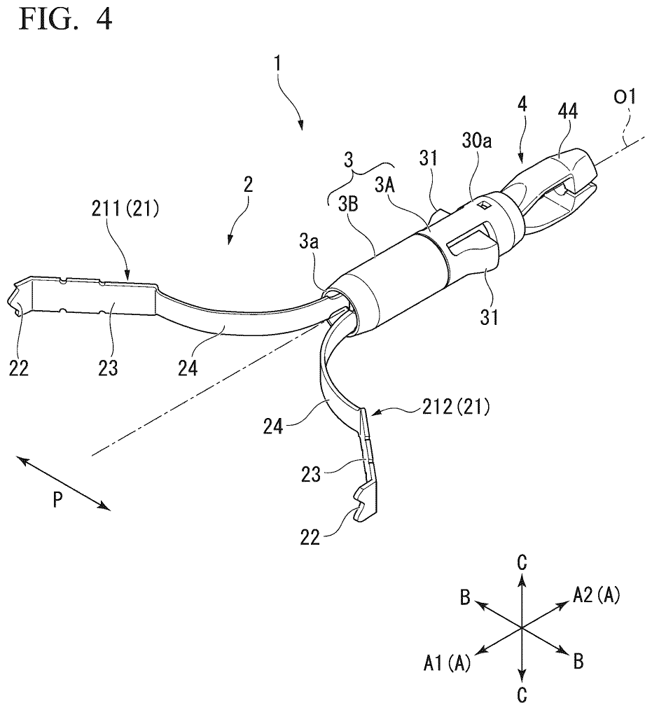

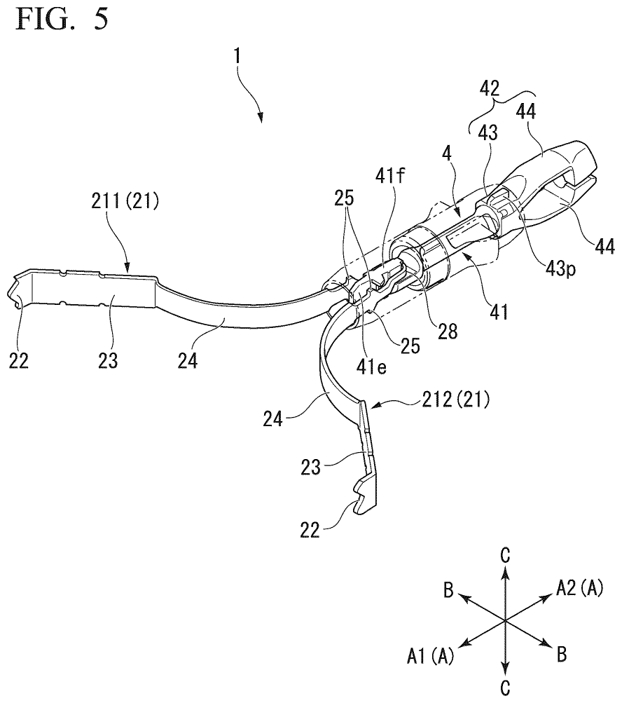

FIG. 4 is a perspective view showing the clip unit 1. FIG. 5 is a perspective view showing the clip unit 1, with the pressing member 3 shown in a see-through state. The clip unit 1 includes a clip 2, a pressing member 3, and a connecting member 4.

In the following description, the clip 2 side in the longitudinal direction A of the clip unit 1 is referred to as the distal end side (distal side) A1 of the clip unit 1, and the connecting member 4 side is referred to as the proximal end side (proximal side) A2 of the clip unit 1. The direction perpendicular to the longitudinal direction A is referred to as the “second direction B” or “left-right direction B”. The direction perpendicular to the longitudinal direction A and the second direction B is referred to as the “first direction C” or “up-down direction C”.

The clip (clip arm) 2 is formed by bending a metal plate at the center. The clip 2 has a pair of arms 21 that can be opened and closed, and a proximal end portion 28 that connects the pair of arms 21. The proximal end side A2 of the clip 2 is inserted into the internal space 38 of the pressing member 3.

The pair of arms 21 includes a first arm 211 and a second arm 212. The first arm 211 and the second arm 212 are arranged on either side of the central axis O1 in the longitudinal direction A of the clip unit 1. The clip 2 may include three or more arms.

The first arm 211 includes, from the distal end A1 to the proximal end A2, a tissue gripping portion 22, a flat gripping portion 23, a sliding portion 24, and an engagement portion 25. The tissue gripping portion 22 is formed by bending the distal end of the first arm 211 inward. The sliding portion 24 is a portion that elastically deforms when the pair of arms 21 are drawn into the pressing member 3.

The second arm 212, like the first arm 211, has a tissue gripping portion 22, a flat gripping portion 23, a sliding portion 24, and an engagement portion 25 from the distal end side A1 to the proximal end side A2.

The engagement portion 25 is a member that can engage with a fastening member 32 of the holding member 3 described later. The distal end side A1 of the engagement portion 25 is formed as a slope that is obtuse with respect to the longitudinal direction A, and the proximal end side A2 is formed as a slope that is acute with respect to the longitudinal direction A.

The proximal end portion 28 connects the first arm 211 and the second arm 212. The first arm 211 and the second arm 212 connected by the proximal end portion 28 are provided so as to be freely opened and closed toward the distal end side A1. The proximal end portion 28 is bent into a U-shape and connected to the hook 41f of the connecting member 4. The proximal end portion 28 is biased so that the pair of arms 21 are in an open state. Therefore, the pair of arms 21 of the clip 2 have a self-expanding force in the opening/closing direction P.

The proximal end portion 28 and the hook 41f of the connecting member 4, which will be described later, are connected by inserting the hook 41f of the connecting member 4 into the proximal end portion 28, which is formed in a U-shape. When the proximal end portion 28 is connected to the connecting member 4, the opening/closing direction P of the pair of arms 21 approximately coincides with the left-right direction B.

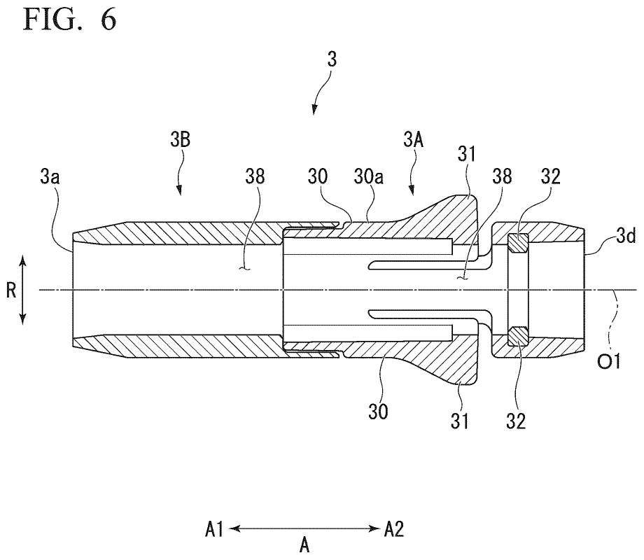

FIG. 6 is a cross-sectional view of the holding member 3 in the longitudinal direction A.

The holding member (tubular member) 3 is a cylindrical member capable of storing at least part of the clip 2. The holding member 3 has an internal space 38 through which the clip 2 advances and retracts in the longitudinal direction A. The holding member 3 can fix the clip 2, which is retracted into the internal space 38, in a closed state. The holding member 3 has a holding tube 3A provided on the proximal end side A2 and a holding pipe 3B provided on the distal end side A1.

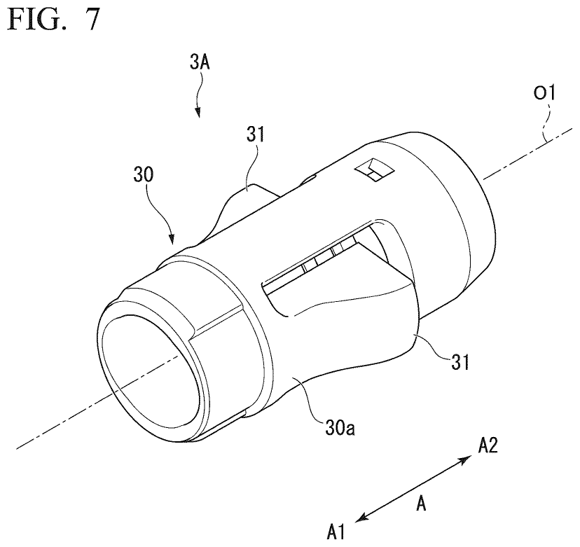

FIG. 7 is a perspective view showing the holding tube 3A.

The holding tube (second tubular member) 3A has a cylindrically formed holding tube main body portion 30, a protruding and recessed wing 31, and a fastening member 32.

The holding tube main body portion 30 is formed by injection molding a material softer than the clip 2, such as a thermoplastic resin having a suitable elasticity, such as PPA (polyphthalamide), PA (polyamide), PEEK (polyether ether ketone), LCP (liquid crystal polymer), etc. The holding tube main body portion 30 may be formed of a metal instead of a thermoplastic resin.

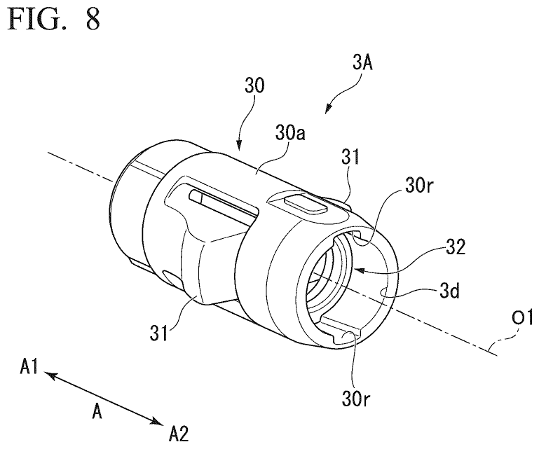

FIG. 8 is a perspective view showing the holding tube 3A as seen from the proximal end side A2.

The proximal end opening 3d of the holding tube main body portion 30 has an engagement recess 30r formed therein to engage with the engagement protrusion 43p of the connecting member 4 described later. The engagement recess 30r is provided on both sides of the central axis O1.

The protruding and retracting wings 31 are a pair of protruding and retracting projections relative to the outer circumferential surface 30a of the holding tube body 30. The protruding and retracting wings 31 are provided on both sides of the central axis O1. The protruding and retracting wings 31 have a basic position in which they protrude outward in the radial direction R relative to the outer circumferential surface 30a. When the protruding and retracting wings 31 receive a force from the outside to the inside in the radial direction R, they become retracted relative to the outer circumferential surface 30a. When the force is released, the protruding and retracting wings 31 return from the retracted state to the protruding state.

The fastening member 32 is a ring-shaped member provided in the internal space 38 of the holding tube 3A. The fastening member 32 is made of metal. The fastening member 32 only needs to be formed to be harder than the holding tube body 30 and may be formed of, for example, a thermoplastic resin instead of a metal.

The clamping pipe (first tubular member) 3B is a cylindrical member made of metal. The clamping pipe 3B is press-fitted into the distal end of the clamping tube 3A. The clamping tube 3A and the clamping pipe 3B may be connected by heat welding, adhesives, or screws.

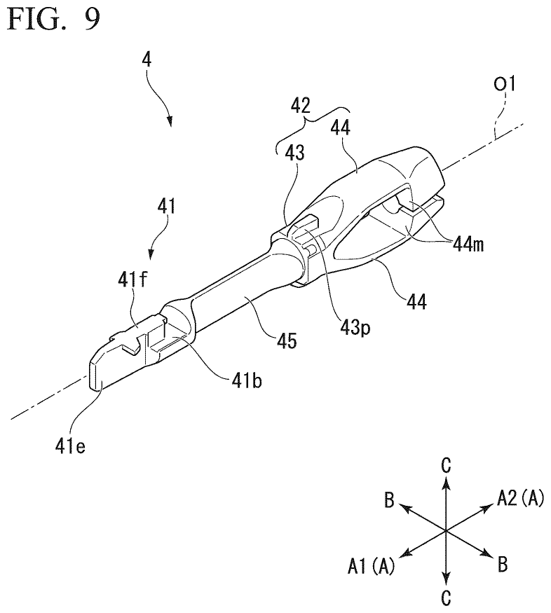

FIG. 9 is a perspective view showing the connecting member 4.

The connecting member 4 is detachably connected to the proximal end portion 28 of the clip 2. The connecting member 4 is also detachably connected to the arrowhead hook 231 that passes through the sheath 220. In other words, the connecting member 4 connects the clip 2 and the arrowhead hook 231. The connecting member 4 includes a distal end connecting portion 41 provided at the distal end, a proximal end connecting portion 42 provided at the proximal end, and a rod-shaped portion 45 that connects the distal end connecting portion 41 and the proximal end connecting portion 42.

The distal end connecting part 41 has a hook 41f at the distal end, a breaking portion 41b provided on the proximal end side A2 of the hook 41f, and a distal end expansion part 41e. The distal end connecting part 41 can be inserted through the internal space 38 of the pressing member 3 when connected to the clip 2.

The hook 41f is a hook that extends in the up-down direction C perpendicular to the central axis O1 and is formed in a substantially cylindrical rod shape. The hook 41f extends from the “lower side” to the “upper side” in the up-down direction C. The proximal end portion 28 of the clip 2 is hooked onto the hook 41f from above.

The breaking portion 41b breaks when a predetermined breaking force is applied to the hook 41f by pulling the proximal end portion 28 to the proximal end side A2. The breaking portion 41b may have a mechanism for releasing the connection between the proximal end portion 28 of the clip 2 and the hook 41f of the connecting member 4. For example, the breaking portion 41b may have a mechanism for releasing the connection between the proximal end portion 28 and the hook 41f by deforming (plastically or elastically) without breaking.

The proximal end connecting portion 42 is an engagement portion with which the arrowhead hook 231 of the clip introduction device 200 is engaged (connected). The proximal end connecting portion 42 has a connecting portion main body portion 43 and a connecting arm 44.

The connecting portion main body portion 43 is provided at the proximal end of the rod-shaped portion 45. The connecting portion main body portion 43 is formed with an engagement protrusion 43p that protrudes in the up-down direction C. The engagement protrusion 43p engages with the engagement recess 30r provided at the proximal end opening of the pressing member 3, thereby restricting the relative rotation of the pressing member 3 and the connecting member 4 in the circumferential direction with respect to the longitudinal direction A.

The connecting arm (a pair of legs) 44 is provided at the proximal end of the connecting body 43 and is bifurcated. The connecting arm 44 is elastically deformable in the vertical direction C relative to the connecting body 43 and can be opened and closed relative to the connecting body 43. The connecting arm 44 is formed with a notch 44m that grips and stores the engaging portion 231a of the arrowhead hook 231. The notch 44m is formed in a shape that fits closely to the outer circumferential surface of the engaging portion 231a of the arrowhead hook 231. The connecting arm 44 can be connected to the arrowhead hook 231 of the clip introduction device 200 when in the open state and can maintain the connection with the arrowhead hook 231 of the clip introduction device 200 when in the closed state.

The rod-shaped portion 45 is a substantially rod-shaped member that extends in the longitudinal direction A. The rod-shaped portion 45 is provided between the distal end connecting portion 41 and the proximal end connecting portion 42 and connects the distal end connecting portion 41 and the proximal end connecting portion 42. The rod-shaped portion 45 can be inserted through the internal space 38 of the pressing member 3.

As shown in FIG. 2, the clip unit 1 is stored in the clip storage area 720 of the cartridge system 700. In a state in which the longitudinal direction A of the clip unit 1 approximately coincides with the height direction H of the cartridge system, the left-right direction B of the clip unit 1 approximately coincides with the width direction W of the cartridge system, and the up-down direction C of the clip unit 1 approximately coincides with the longitudinal direction L of the cartridge system, the clip unit 1 is stored in the clip storage area 720.

In the clip unit 1 stored in the clip storage area 720, the clip 2 on the distal end side A1 is disposed on the lower side in the height direction H, and the connecting member 4 on the proximal end side A2 is disposed on the upper side in the height direction H. At least the proximal end of the connecting member 4 is disposed in the sheath insertion area 740.

### [Method of Loading Clip Unit 1]

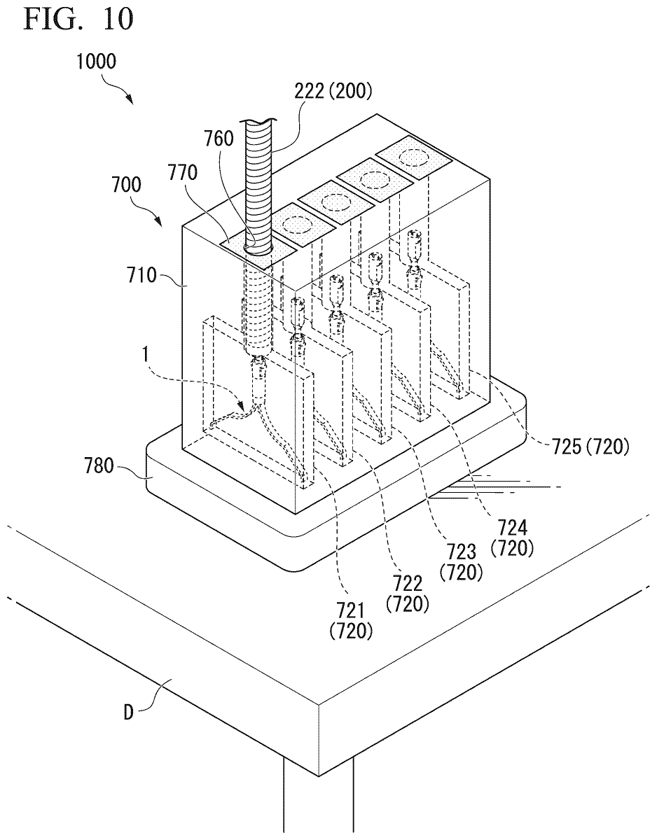

Next, the operation of the clip system 1000 will be described. FIGS. 10 to 14 are diagrams explaining a method of loading the clip unit 1 into the clip introduction device 200 using the cartridge system 700.

FIG. 10 shows the sheath 220 inserted into the clip storage area 720.

The user presses the sheath 220 of the clip introduction device 200 against the seal 770 to break the seal 770 and inserts the sheath 220 into the sheath insertion area 740 from the opening 760. The clip storage area 720 into which the sheath 220 has been inserted is released from the sterile state. The user further inserts the sheath 220 and brings the distal chip 221 of the sheath 220 close to the connecting member 4 of the clip unit 1.

The cartridge system 700 stands on its own with the opening 760 located on the upper side in the height direction H. Therefore, when inserting the sheath 220 into the cartridge system 700, the user does not need to hold and fix the cartridge system 700 by hand. This allows the user to efficiently load the clip unit 1 with less effort.

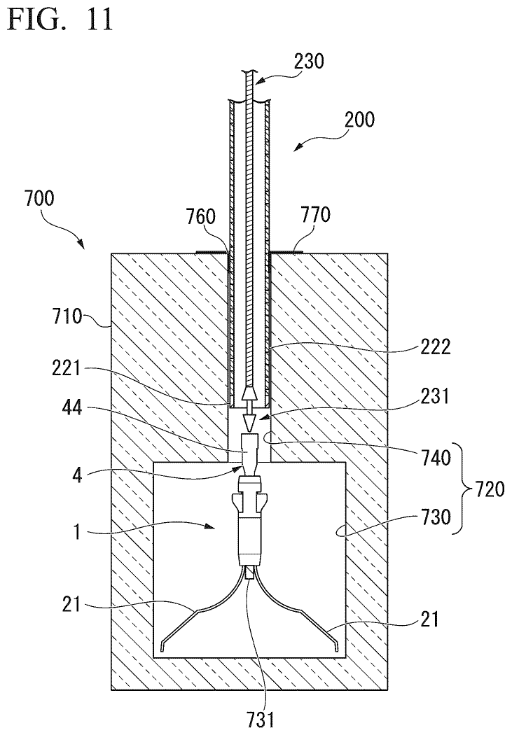

FIG. 11 shows the clip unit 1 before loading.

The user advances the slider 242 relative to the operation portion body 241 of the operation portion 240 and advances the operation wire 230 relative to the sheath 220, thereby bringing the arrowhead hook 231 closer to the connecting member 4.

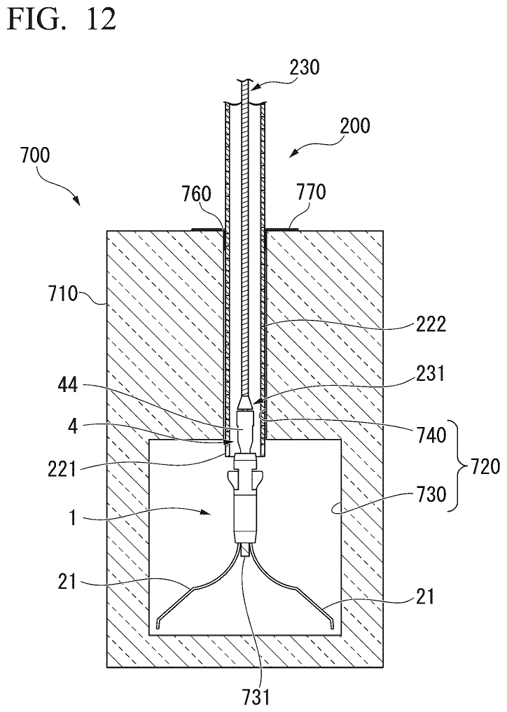

FIG. 12 shows the clip unit 1 after loading.

The user advances the slider 242 relative to the operation portion body 241 of the operation portion 240 and inserts the arrowhead hook 231 into the connecting member 4. The clip unit 1 engages with a stopper 731 provided in the clip storage area 720 and is restricted from moving downward in the height direction H.

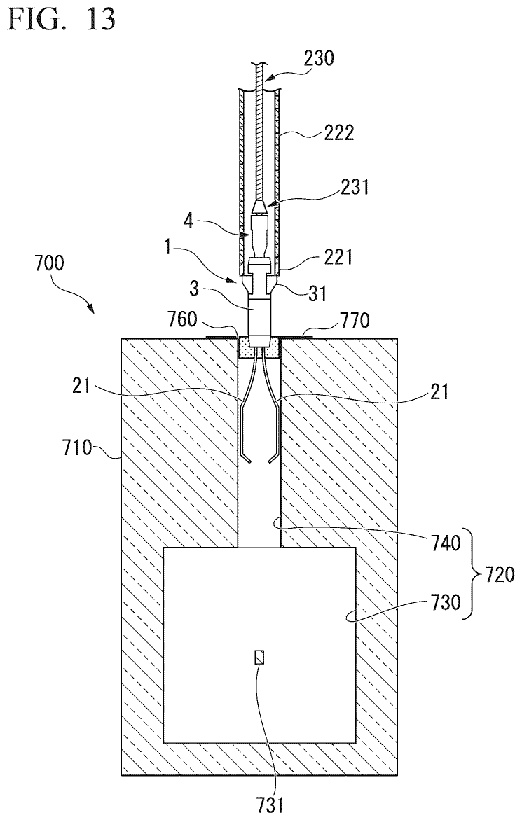

FIG. 13 shows the sheath 220 being pulled out of the clip storage area 720.

The user pulls out the sheath 220 from the clip storage area 720. The pair of arms 21 temporarily close.

Since the retractable wings 31 of the clip unit 1 engage with the distal chip 221, the clip unit 1 is not pulled into the sheath 220. The user may retract the clip unit 1 into the sheath 220 by setting the retractable wings 31 to the retracted state.

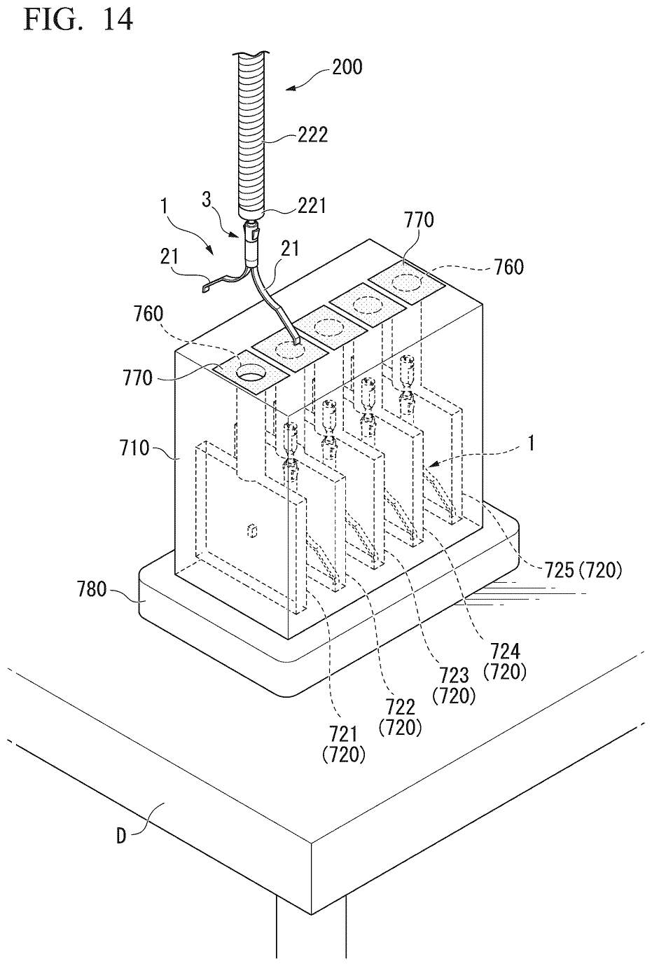

FIG. 14 shows the clip introduction device 200 with the clip unit 1 loaded.

The clip unit 1 stored in the first clip storage area 721 is loaded into the clip introduction device 200. A seal 770 is attached to the opening 760 of the second clip storage area 722 etc. in which an unused clip unit 1 that has not been removed from the cartridge system 700 is stored, and the second clip storage area 722 etc. in which an unused clip unit 1 is stored are maintained in a sterile state. Therefore, the user can use an unused clip unit 1 among the multiple clip units 1 stored in the cartridge system 700 for a different case.

The method of loading the clip unit 1 into the clip introduction device 200 may be the following method. The user advances the slider 242 relative to the operation portion main body portion 241 of the operation portion 240 and presses the clip introduction device 200 against the seal 770 with the distal end (arrowhead hook 231) of the operation wire 230 protruding from the sheath 220. The user breaks the seal 770 with the distal end (arrowhead hook 231) of the operation wire 230. The user inserts the sheath 220 into the sheath insertion area 740 from the opening 760. Next, with the distal end of the operation wire 230 (arrowhead hook 231) protruding from the sheath 220, the user advances the clip introduction device 200 relative to the cartridge system 700 to insert the arrowhead hook 231 into the connecting member 4.

### [Operation and Function of Clip Unit 1]

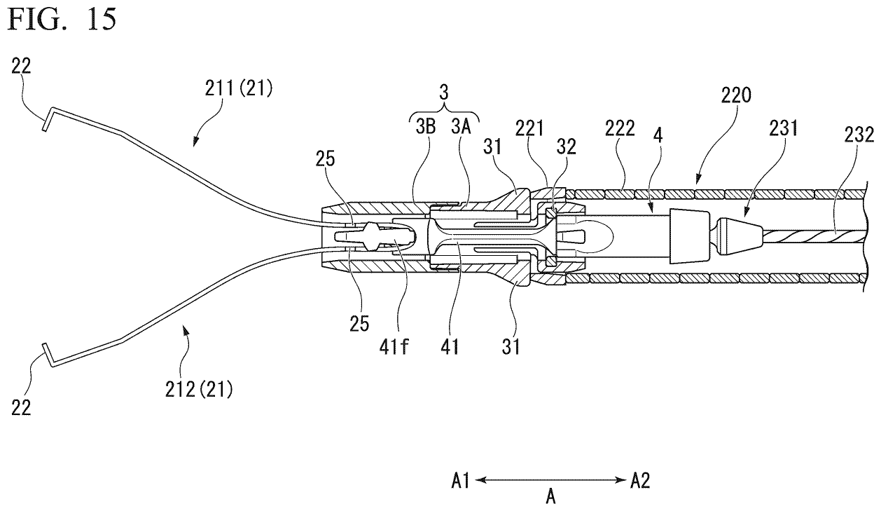

Next, the operation and function of the clip unit 1 will be described with reference to FIGS. 15 to 18.

FIG. 15 is a diagram showing the clip unit 1 introduced into the body.

The surgeon introduces the clip unit 1 loaded in the sheath 220 into the body via the channel of the endoscope. Next, the surgeon advances the slider 242 along the operation portion main body portion 241 to advance the arrowhead hook 231. The surgeon advances the clip unit 1 until the protruding and retracting wing 31 comes out of the sheath 220. By coming out of the sheath 220, the protruding and retracting wing 31 returns from the retracted state to the protruding state, which is the basic position.

When the distal end of the pair of arms 21 comes out of the sheath 220, the self-expanding force of the pair of arms 21 serves as a restoring force, and the clip 2 returns to the open state while moving toward the distal end relative to the holding member 3. Even when the pair of arms 21 return to the open state and protrude most from the holding member 3, the engagement portion 25 is disposed in the internal space 38 of the holding member 3.

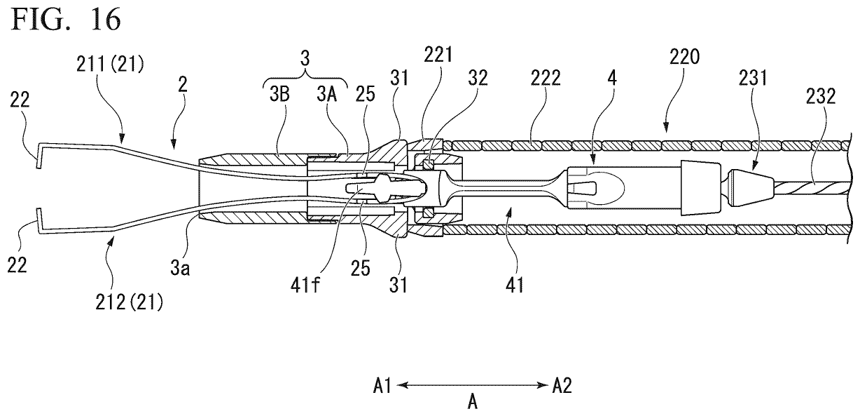

FIG. 16 is a diagram showing the clip unit 1 with the pair of arms 21 closed.

The surgeon retracts the arrowhead hook 231 by retracting the slider 242 along the operation portion main body portion 241. The connecting member 4 connected to the arrowhead hook 231 pulls the clip 2. The pair of arms 21, which have a self-expanding force, are pulled toward the proximal end side A2, and thus push the distal opening 3a of the holding member 3 toward the proximal end side A2. The protruding and retracting wings 31, which are in the protruding state, are engaged with the sheath 220 and are therefore not retracted into the sheath 220. Therefore, the clip 2 pulled by the connecting member 4 is drawn into the holding member 3.

When the proximal end portion 28 of the clip 2 is pulled toward the proximal end side A2 of the holding member 3 by the connecting member 4, the pair of arms 21 are drawn into the holding member 3 and the pair of arms 21 gradually close. When the pulling force on the proximal end portion 28 is released in this state, the clip 2 returns to the open state while moving toward the distal end side A1, using the self-expanding force of the pair of arms 21 as a restoring force. The surgeon can return the pair of arms 21 to the open state and re-grasp the tissue.

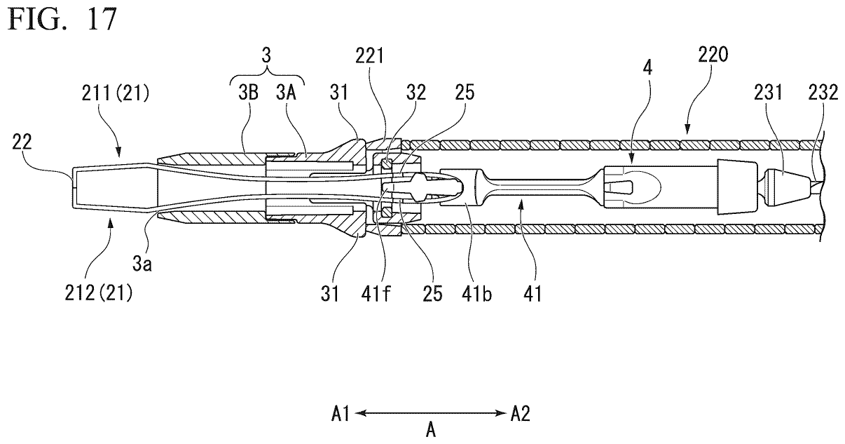

FIG. 17 shows the clip unit 1 with the clip 2 locked.

The proximal end portion 28 is further pulled toward the proximal end side A2 of the pressing member 3, so that the engaging portion 25 is pulled toward the proximal end side A2 from the fastening member 32. The proximal end side A2 of the engaging portion 25 is formed as an obtuse-angled inclined surface, so that the engaging portion 25 can be easily pulled toward the proximal end side from the fastening member 32. On the other hand, the distal end side A1 of the engaging portion 25 is formed as an acute-angled inclined surface, so that when the engaging portion 25 is pulled toward the proximal end side A2 from the fastening member 32, the engaging portion 25 and the fastening member 32 engage with each other. As a result, the movement of the clip 2 toward the distal end side A1 relative to the pressing member 3 is restricted, and the pair of arms 21 are locked in the closed state. When the pair of arms 21 are locked in the closed state, the pair of arms 21 cannot return to the open state.

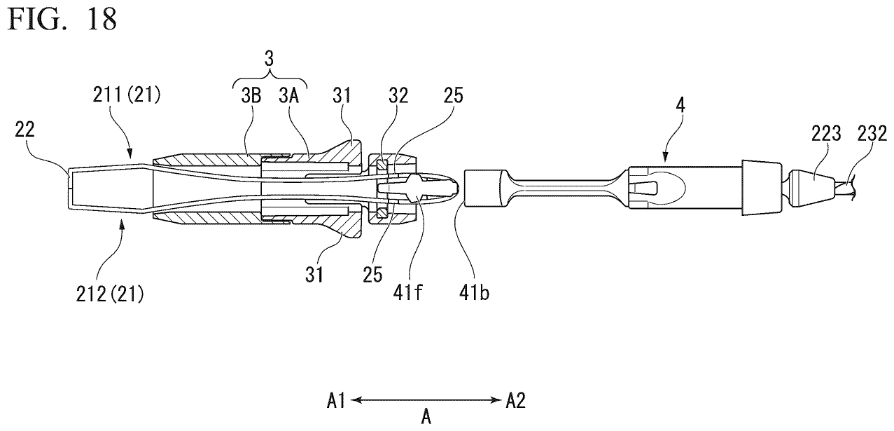

FIG. 18 shows the clip unit 1 with the clip 2 separated.

The surgeon further pulls the clip 2. A predetermined amount of breaking force is applied to the hook 41f by pulling, and the breaking portion 41b breaks. The breaking strength of the breaking portion 41b is lower than that of the connecting portion body 43. Therefore, the breaking portion 41b breaks, not the connecting portion body 43.

The surgeon reloads a new clip unit 1 into the clip introduction device 200 using the cartridge system 700.

### [Method of Storing the Clip Unit 1]

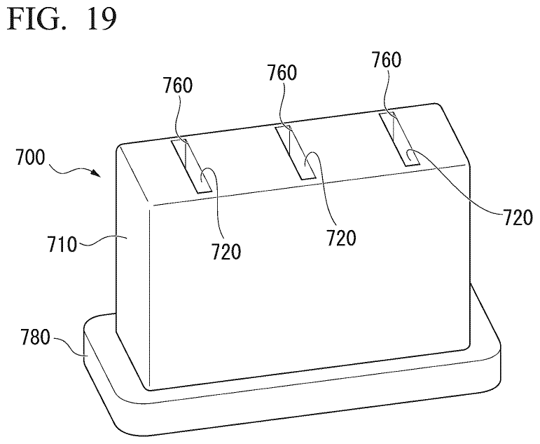

FIG. 19 is a diagram showing an example of an implementation form of the cartridge system.

As shown in FIG. 19, the opening 760 may have a shape that allows the clip unit 1 with the clip 2 in an open state to be inserted. In this case, the clip unit 1 can be stored in the clip storage area 720 through the opening 760 before the seal 770 is attached to the opening 760. In addition, the clip unit 1 loaded in the sheath 220 can be taken out from the opening 760 with the clip 2 in an open state.

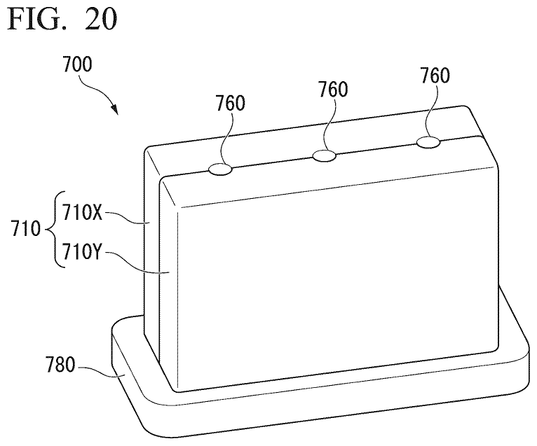

FIG. 20 shows another example of the implementation of the cartridge system.

The main body portion 710 may be shaped so that the clip storage area 720 is exposed when part of the main body portion 710 is removed. The main body portion 710 shown in FIG. 20 is composed of a right main body portion 710X and a left main body portion 710Y, and either one can be removed. In this case, when part of the main body portion 710 is removed from the main body portion 710, the clip unit 1 can be stored in the clip storage area 720. In order to maintain the sterility of the clip storage area 720, the connection between the right main body portion 710X and the left main body portion 710Y may be sealed.

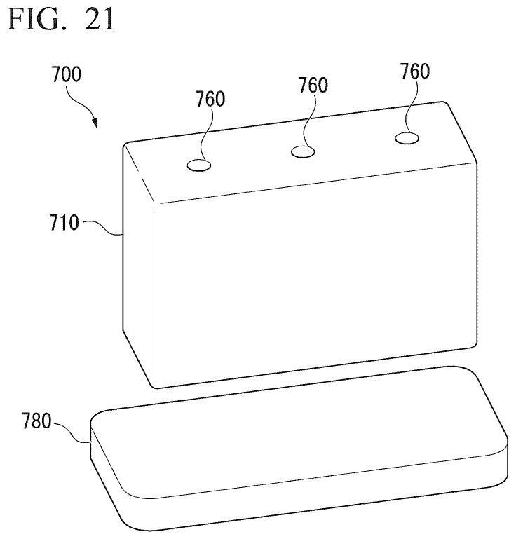

FIG. 21 shows another example of the implementation of the cartridge system.

The main body portion 710 and the base 780 are separable, and when the main body portion 710 is removed from the base 780, the clip storage area 720 may be exposed. In this case, when the main body portion 710 and the base 780 are separated, the clip unit 1 can be stored in the clip storage area 720.

According to the clip system 1000 according to this embodiment, among the multiple clip units 1 stored in the cartridge system 700, unused clip units 1 can be used for different cases.

The first embodiment of the present disclosure has been described above in detail with reference to the drawings, but the specific configuration is not limited to this embodiment, and design changes and the like within the scope of the gist of the present disclosure are also included. In addition, the components shown in the above-mentioned embodiment and the modified examples shown below can be appropriately combined to configure the configuration.

### (Modification 1-1)

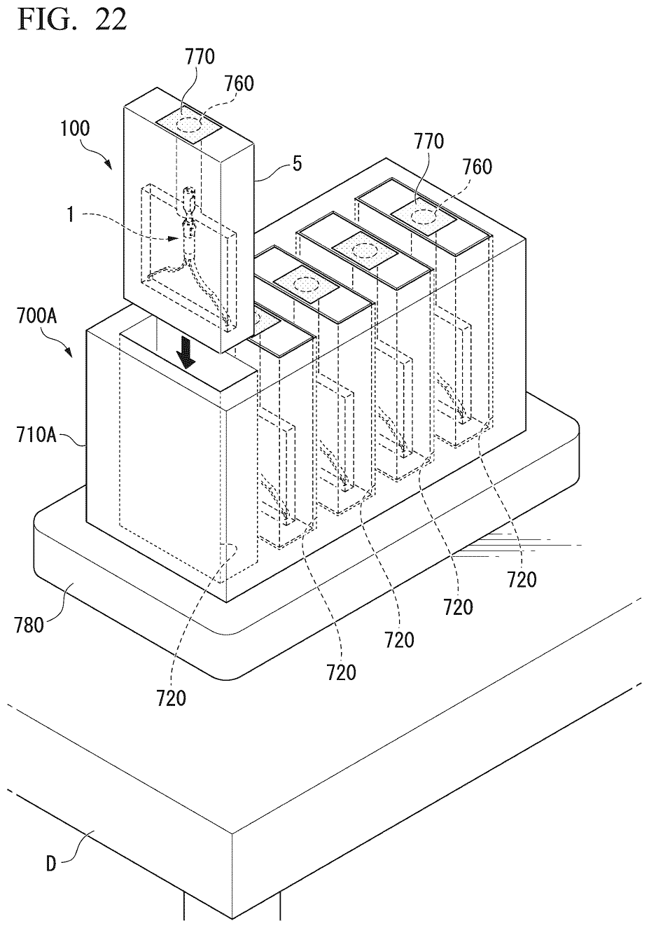

FIG. 22 is a diagram showing a cartridge system 700A, which is a modified example of the cartridge system 700. The cartridge system 700A stores a cartridge unit 100 in which a clip unit 1 is stored, rather than a single clip unit 1, in the clip storage area 720. In other words, the clip storage area 720 may store a cartridge unit 100 in which a clip unit 1 is stored, rather than a single clip unit 1.

The cartridge unit 100 has a clip unit 1 and a cartridge 5 that stores the clip unit 1 so that it can be loaded. The cartridge unit 100 has an opening 760 and a cover 770. When the cartridge unit 100 is stored in the clip storage area 720, the opening 760 is located on the upper side in the height direction H. As in the above embodiment, the user does not need to hold and fix the cartridge unit 100 by hand when inserting the sheath 220 into the cartridge unit 100 stored in the cartridge system 700A. This allows the user to efficiently load the clip unit 1 with less effort.

### (Modification 1-2)

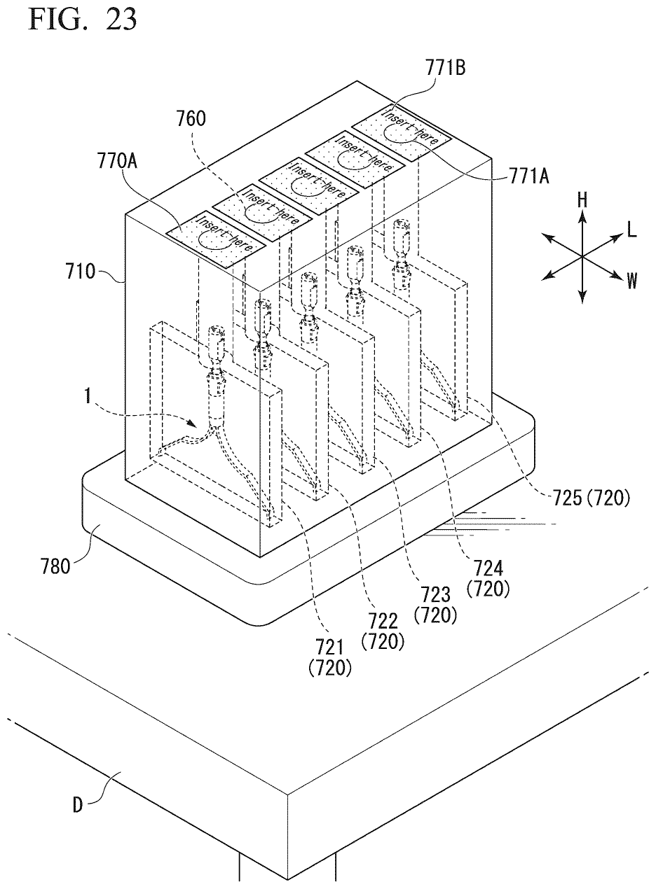

FIG. 23 shows a seal 770A, which is a modification of the seal 770.

The seal 770A has a mark 771 that indicates the position where the clip introduction device 200 breaks through the seal 770A. The mark 771 is, for example, a mark FIG. 771A that indicates the breakthrough position in the seal 770A. The mark FIG. 771A illustrated in FIG. 23 is a circle drawn on the sticker 770A. The mark 771 is, for example, guide text information 771B that guides the position of breaking through the sticker 770A. The guide text information 771B illustrated in FIG. 23 is text information “Insert here” that guides the user in the operation. The mark 771 may be provided on the main body portion 710, not on the sticker 770A.

### Second Embodiment

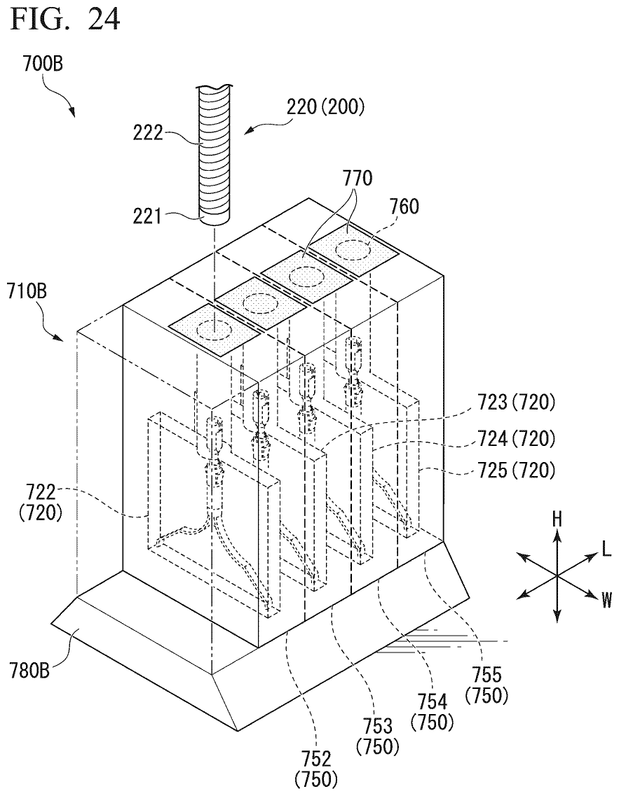

A second embodiment of the present disclosure will be described with reference to FIG. 24. In the following description, the same reference numerals will be used to designate components that are common to those already described and duplicate descriptions will be omitted.

FIG. 24 is a diagram showing a cartridge system 700B according to this embodiment.

The cartridge system 700B can stand on its own on a mounting surface such as a workbench D as shown in FIG. 1. Specifically, the cartridge system 700B includes a main body portion 710B in which a plurality of clip units 1 are stored, and a base 780B disposed on the mounting surface.

The main body portion 710B has a plurality of main body portions 750. The main body portion 710B illustrated in FIG. 24 has five main body portions 750 (a first main body portion 751, a second main body portion 752, a third main body portion 753, a fourth main body portion 754, and a fifth main body portion 755).

Each of the plurality of main body portions 750 has a clip storage area 720. The first main body portion 751 has a first clip storage area 721. The second main body portion 752 has a second clip storage area 722. The third main body portion 753 has a third clip storage area 723. The fourth main body portion 754 has a fourth clip storage area 724. The fifth main body portion 755 is formed with a fifth clip storage area 725. In FIG. 24, the first main body portion 751 has been separated and discarded.

Each of the multiple clip storage areas 720 is provided with an opening 760 into which the sheath 220 of the clip introduction device 200 can be inserted, and a lid 770 that closes the opening 760 in an independently releasable manner. The opening 760 is connected to the sheath insertion area 740. When the opening 760 is closed by the lid 770, the clip storage area 720 is maintained in a sterile state.

The base 780B is a flat member that is disposed on a placement surface such as the workbench D. The base 780B supports each of the multiple main body portions 750 in an independently separable manner. The base 780B supports the main body portion 750 with the opening 760 positioned at a position where the sheath 220 of the clip introduction device 200 can be inserted. The base 780B illustrated in FIG. 24 supports the main body portion 750 with the opening 760 located on the upper side in the height direction H.

The user can separate the main body portion 750 loaded with the clip unit 1 from the base 780B and discard it. The opening 760 of the other main body portion 750 in which the unused clip unit 1 that has not been removed from the cartridge system 700B is stored is provided with a seal 770, and the clip storage area 720 is maintained in a sterile state. Therefore, the user can use the unused clip unit 1 among the multiple clip units 1 stored in the cartridge system 700B for a different case.

According to the cartridge system 700B according to this embodiment, the unused clip unit 1 among the multiple clip units 1 stored in the cartridge system 700B can be used for a different case. The main body portion 750 in which the clip unit 1 has been used can be separated from the base 780B and discarded. Therefore, the user will not mistakenly insert the sheath 220 into a main body portion in which the clip unit 1 is not stored.

The second embodiment of the present disclosure has been described above in detail with reference to the drawings, but the specific configuration is not limited to this embodiment, and design modifications within the scope of the present disclosure are also included. In addition, the components shown in the above-mentioned embodiment and the modified examples shown below can be appropriately combined to configure the present disclosure.

### (Modification 2-1)

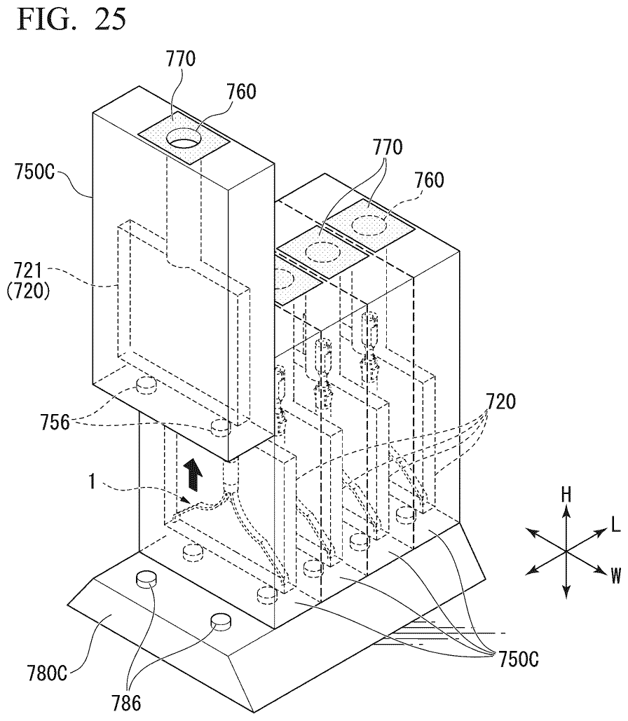

FIG. 25 is a diagram showing a main body portion 750C, which is a modified example of the main body portion 750, and a base 780C, which is a modified example of the base 780B. The main body portion 750C has a recess 756 on the lower surface. The base 780C has a protrusion 786 on the upper surface. The recess 756 fits into the protrusion 786, thereby connecting the main body portion 750C to the base 780C. The user can easily remove the main body portion 750C from the base 780C.

### (Modification 2-2)

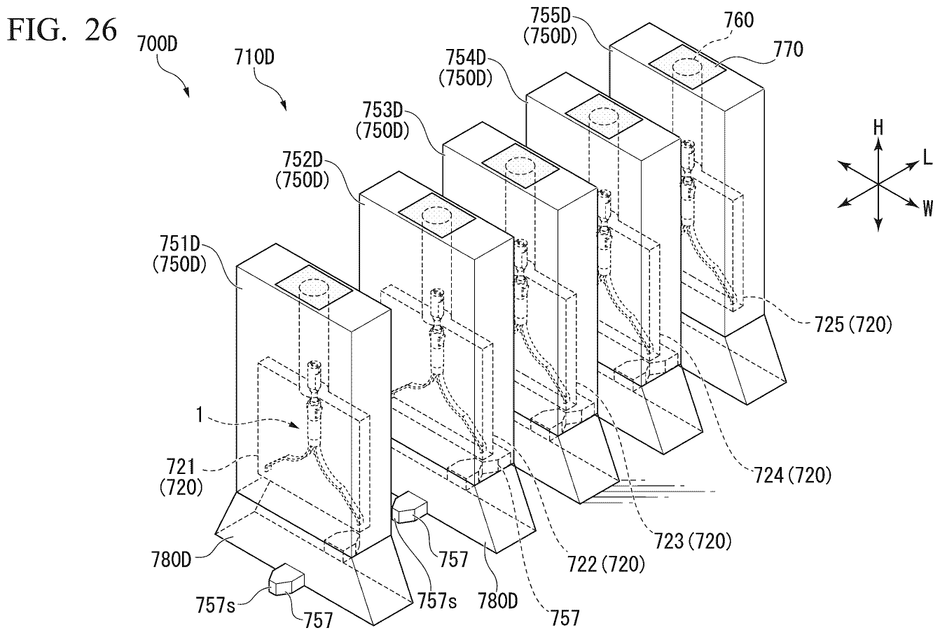

FIG. 26 is a diagram showing a cartridge system 700D, which is a modified example of the cartridge system 700B. The cartridge system 700D includes a main body portion 710D. The main body portion 710D has a plurality of main body portions 750D. The cartridge system 700D illustrated in FIG. 26 has five main body portions 750D (a first main body portion 751D, a second main body portion 752D, a third main body portion 753D, a fourth main body portion 754D, and a fifth main body portion 755D). A clip storage area 720 is formed in each of the plurality of main body portions 750D. The plurality of main body portions 750D are arranged along the longitudinal direction L. Adjacent main body portions 750D are connected via a connecting part 757. The connecting part 757 has a narrowed constricted part 757s. By cutting off the constricted part 757s of the connecting part 757, the adjacent main body portions 750D can be separated.

Each of the plurality of main body portions 750D has a base 780D. The base 780D supports the main body portion 750D with the opening 760 located at a position where the sheath 220 of the clip introduction device 200 can be inserted. The base 780B illustrated in FIG. 26 supports the main body portion 750D with the opening 760 located at the upper side in the height direction H. The connecting portion 757 is provided on the base 780D.

### (Modification 2-3)

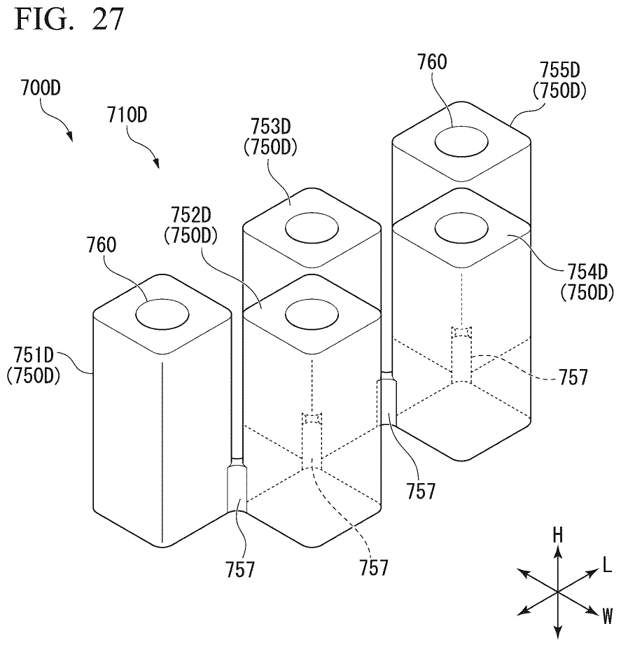

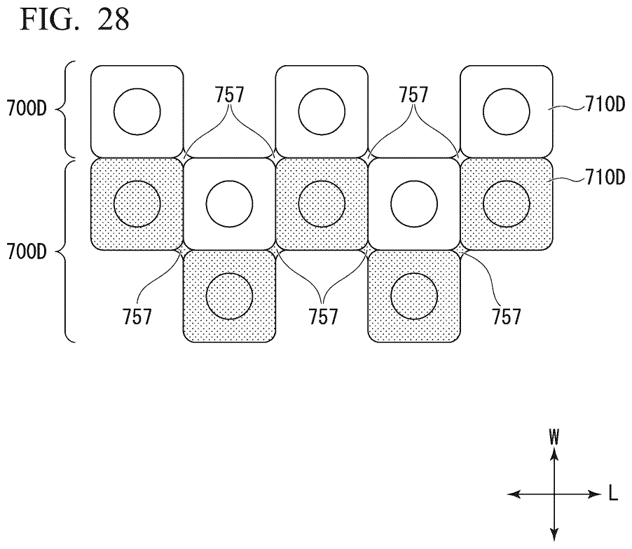

FIGS. 27 and 28 are views showing another embodiment of the cartridge system 700D.

The main body portion 750D may be formed in a rectangular parallelepiped shape as shown in FIG. 26. The multiple main body portions 750D are connected in a zigzag shape when viewed from above in the height direction H. Therefore, the multiple main body portions 750D have high self-supporting stability even without a base. Although the cartridge system 700D has a zigzag shape, as shown in FIG. 28, multiple cartridge systems 700D can be stacked and stored, so that the storage capacity in a carton is not reduced.

### (Modification 2-4)

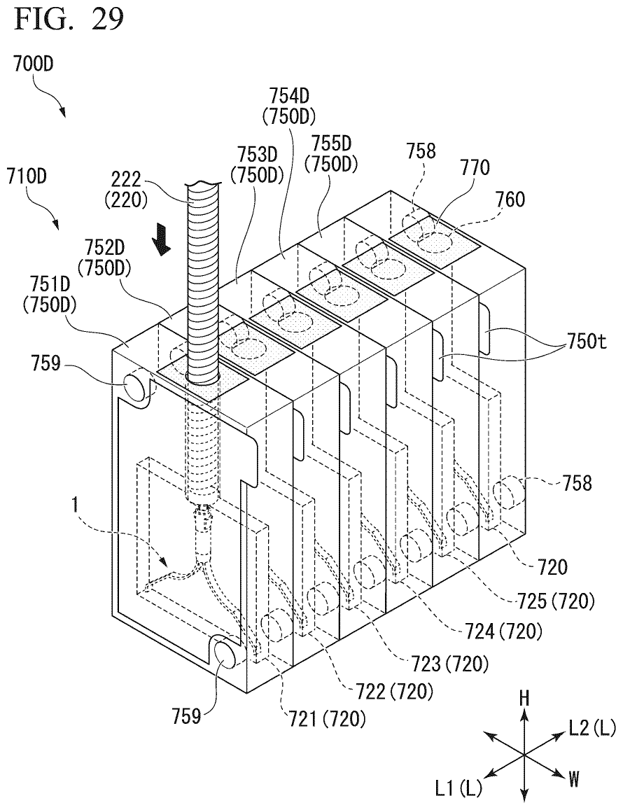

FIGS. 29 to 31 are diagrams showing another embodiment of the cartridge system 700D.

As shown in FIG. 29, adjacent main body portions 750D may be detachably connected by bosses 758 and holes 759. The holes 759 are recessed portions provided on the front side L1 (one side in the longitudinal direction L) of the main body portion 750D. The holes 759 are recessed on the rear side L2 (the other side in the longitudinal direction L). The bosses 758 are protruding portions provided on the rear side L2 of the main body portion 750D. The bosses 758 protrude to the rear side L2. The bosses 758 and the holes 759 fit together to detachably connect the main body portions 750D adjacent in the longitudinal direction L.

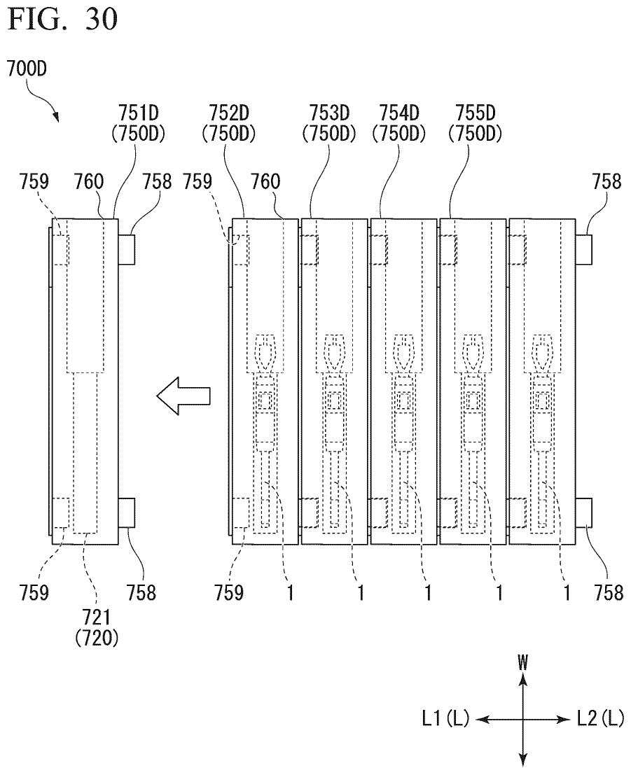

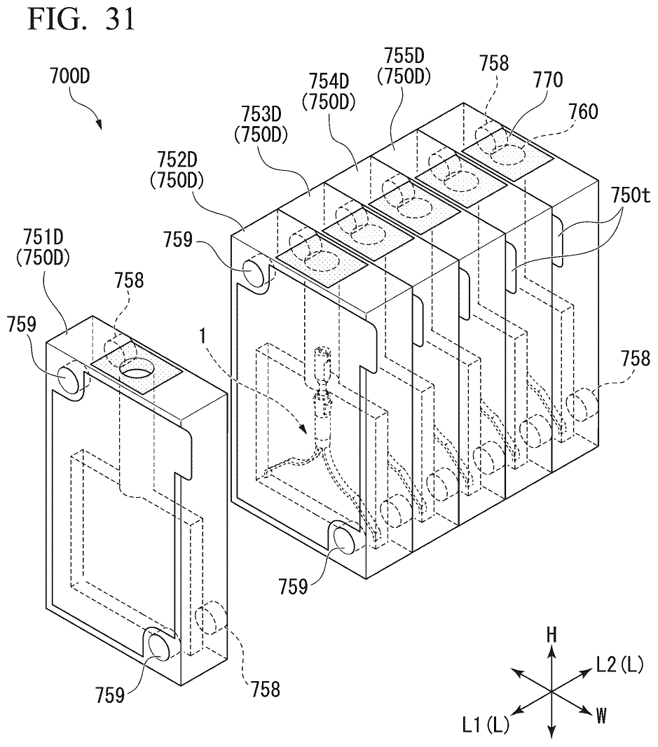

As shown in FIGS. 30 and 31, the user can separate the main body portion 750D with the clip unit 1 loaded from the unused main body portion 750D and discard it. The main body portion 750D has a tab 750t that protrudes in the width direction W. Therefore, the user can easily separate the adjacent main body portion 750D by pulling the tab 750t.

Each main body portion 750D functions as a cartridge unit 100 in which the clip unit 1 can be loaded. The user may separate the main body portion 750D with the clip unit 1 stored therein before loading the clip unit 1, rather than after loading the clip unit 1. The user can use the main body portion 750D with the clip unit 1 stored therein as the cartridge unit 100 to load the clip unit 1. The user can also connect the separated main body portion 750D to the unused main body portion 750D in cases where the separated main body portion 750D has not been used. That is, the user can connect unused main body portions 750D and store them together and use them for different cases.

As with the cartridge system 700A shown in FIG. 22, the clip storage area 720 of the main body portion 750D may store not only a single clip unit 1 but also a cartridge unit 100 in which a clip unit 1 is stored.

### Third Embodiment

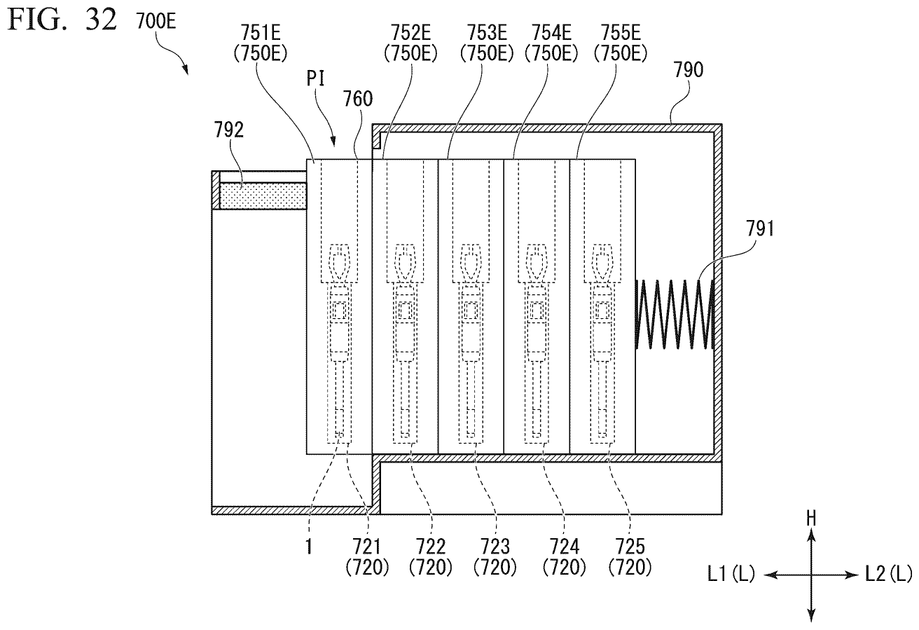

A third embodiment of the present disclosure will be described with reference to FIGS. 32 to 35. In the following description, the same reference numerals will be used to designate components that are common to those already described, and redundant description will be omitted.

FIG. 32 is a diagram showing a cartridge system 700E according to this embodiment.

The cartridge system 700E includes a main body portion 710E in which multiple clip units 1 are stored, and a storage case 790.

The main body portion 710E has multiple main body portions 750E. The cartridge system 700E illustrated in FIG. 32 has five main body portions 750E (first main body portion 751E, second main body portion 752E, third main body portion 753E, fourth main body portion 754E, fifth main body portion 755E). Each of the multiple main body portions 750E is formed with a clip storage area 720. The multiple main body portions 750E are arranged along the longitudinal direction L.

Each of the multiple clip storage areas 720 is provided with an opening 760 into which the sheath 220 of the clip introduction device 200 can be inserted, and a lid 770 that independently closes the opening 760 in an openable manner. The opening 760 communicates with the sheath insertion area 740. When the opening 760 is closed by the lid 770, the clip storage area 720 is maintained in a sterile state. Note that the lid 770 is not illustrated in FIG. 32 and other figures.

The storage case 790 is a case that stores a plurality of main body portions 750E so that they can be discharged in sequence. The storage case 790 has a spring 791 that pushes out the plurality of main body portions 750E. The spring 791 pushes out the plurality of main body portions 750E arranged along the longitudinal direction L from the rear side L2 toward the front side L1. The plurality of main body portions 750E pushed out toward the front side L1 by the spring 791 engage with a stopper 792 provided on the front side L1 of the storage case 790.

Of the plurality of main body portions 750E pushed out toward the front side L1 by the spring 791, only the main body portion 750E that is located furthest forward on the front side L1 and engages with the stopper 792 is exposed from the storage case 790. Of the plurality of main body portions 750E shown in FIG. 32, only the first main body portion 751E is exposed from the storage case 790. The second body portion 752E, the third body portion 753E, the fourth body portion 754E and the fifth body portion 755E are not exposed from the storage case 790.

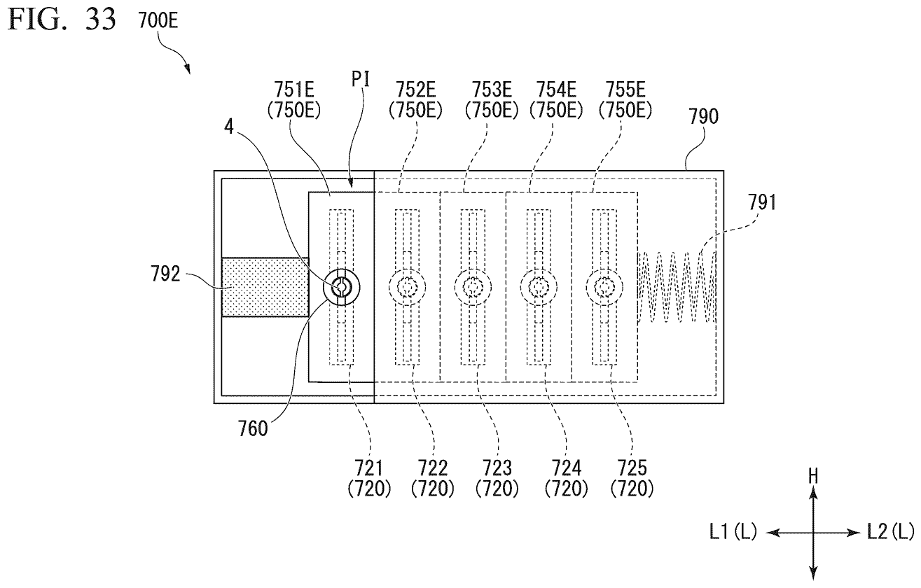

FIG. 33 is a diagram showing the first body portion 751E arranged at the insertable position PI.

The body portion 750E exposed from the storage case 790 is arranged at the insertable position PI where the sheath 220 of the clip introduction device 200 can be inserted. Of the multiple body portions 750E shown in FIG. 33, only the first body portion 751E is exposed from the storage case 790 and arranged at the insertable position PI. The second body portion 752E, the third body portion 753E, the fourth body portion 754E and the fifth body portion 755E are not exposed from the storage case 790 and are not arranged at the insertable position PI.

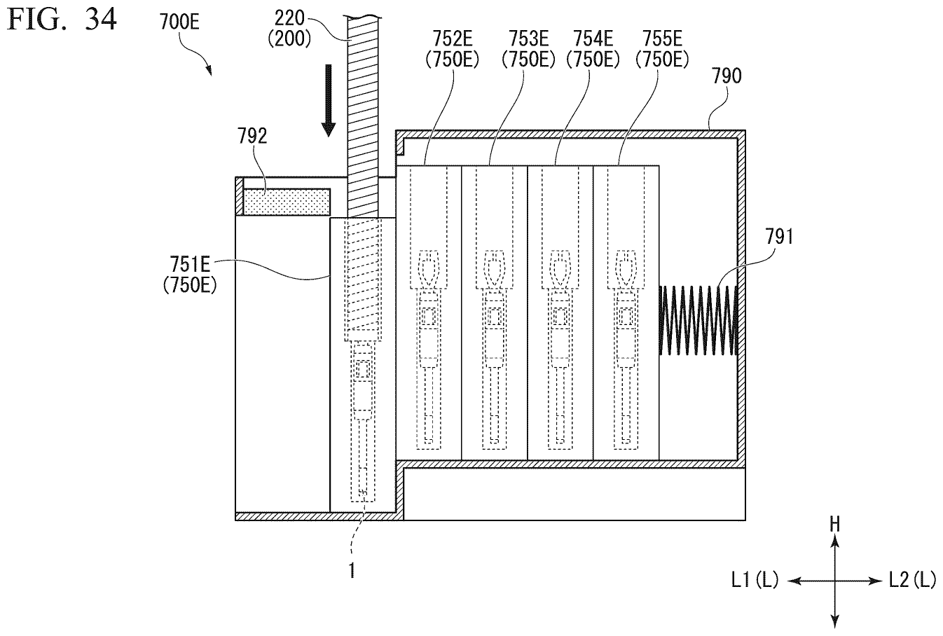

FIG. 34 is a diagram showing the body portion 750E into which the sheath 220 is inserted.

The user can insert the sheath 220 into the opening 760 of the main body portion 750E arranged at the insertable position PI. The main body portion 750E with the sheath 220 inserted moves downward and is no longer engaged with the stopper 792.

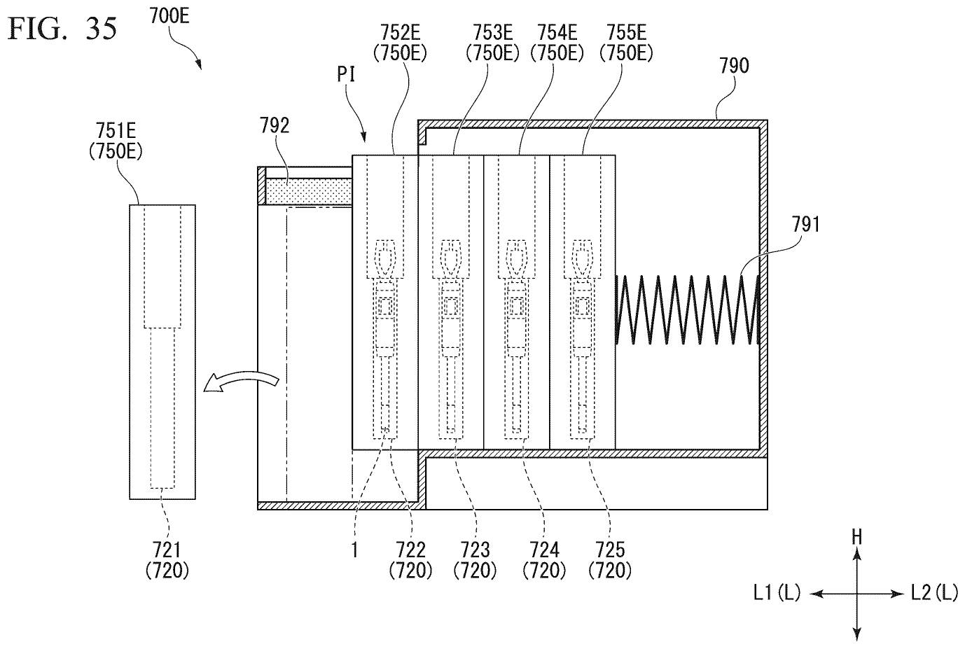

FIG. 35 shows the main body portion 750E with the sheath 220 pulled out.

After loading the clip unit 1, the user pulls out the sheath 220 from the main body portion 750E arranged at the insertable position PI. The main body portion 750E with the sheath 220 pulled out does not engage with the stopper 792 and is ejected to the outside from the storage case 790. In the multiple main body portions 750E shown in FIG. 35, the first main body portion 751E with the sheath 220 pulled out is ejected from the storage case 790. The second main body portion 752E arranged at the rear side L2 of the first main body portion 751E is arranged at the insertable position PI. This operation is then repeated.

According to the cartridge system 700E according to this embodiment, among the multiple clip units 1 stored in the cartridge system 700B, an unused clip unit 1 can be used for a different case. Since one main body portion 750E in which a clip unit 1 can be loaded is disposed at the insertable position PI in which the sheath 220 can be inserted, erroneous insertion by the user does not occur.

The third embodiment of the present disclosure has been described above in detail with reference to the drawings, but the specific configuration is not limited to this embodiment, and design changes and the like within the scope of the gist of the present disclosure are also included. In addition, the components shown in the above-mentioned embodiment and the modified examples shown below can be appropriately combined to form a configuration.

### Fourth Embodiment

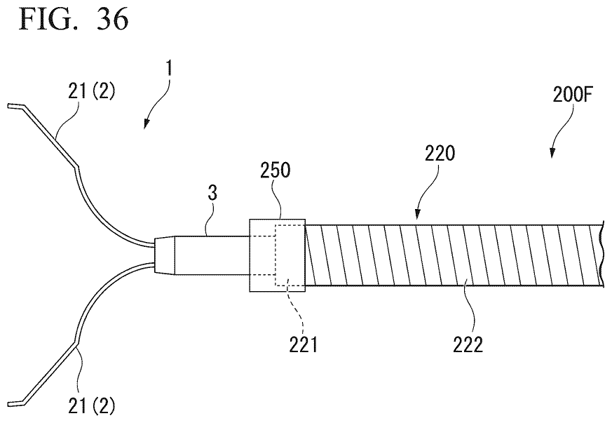

A fourth embodiment of the present disclosure will be described with reference to FIGS. 36 to 43. In the following description, the same reference numerals are used for configurations that are common to those already described, and duplicated description will be omitted.

### [Clip System 1000F]

The clip system 1000F according to the fourth embodiment of the present disclosure includes a cartridge system 700F and a clip introduction device (applicator) 200F.

FIG. 36 is a diagram showing the clip introduction device 200F.

The clip introduction device (applicator) 200F differs from the clip introduction device 200 of the first embodiment in that it is connected to the clip unit 1 using a connecting member 250 instead of the arrowhead hook (connecting portion) 231.

The connecting member (coupler) 250 is formed in a cylindrical shape. The connecting member 250 covers and connects the clip unit 1 and the distal chip (distal end portion) 221 of the sheath 220 from the outside.

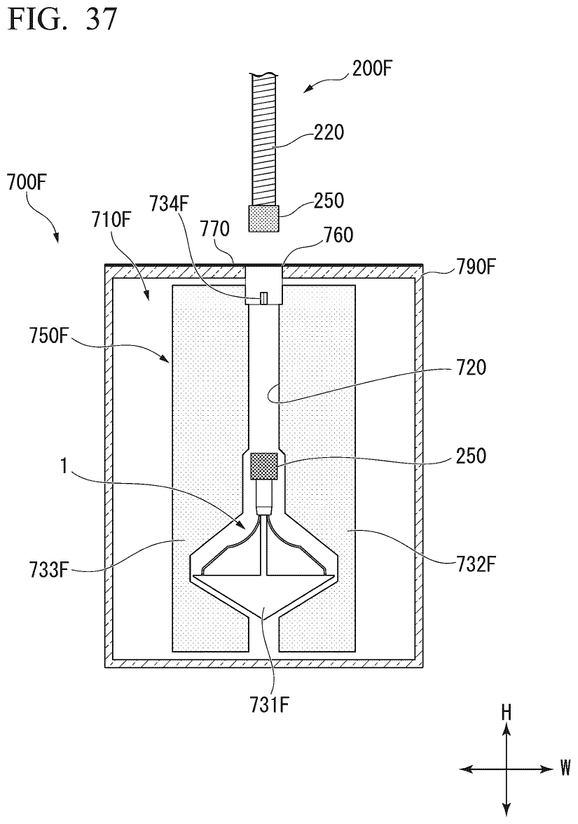

FIG. 37 is a diagram showing the cartridge system 700F.

The cartridge system 700F includes a main body portion 710F in which multiple clip units 1 are stored, and a storage case 790F.

The main body portion 710F includes multiple main body portions 750F. In the cartridge system 700F illustrated in FIG. 37, only one main body portion 750F is shown. A clip storage area 720 is formed in each of the multiple main body portions 750F.

The main body portion 750F includes a stopper 731F, a right main body portion 732F, a left main body portion 733F, and a sharp member 734F.

The right main body portion 732F and the left main body portion 733F are members that sandwich the clip unit 1 from both sides in the width direction W and form the clip storage area 720 in the sandwiched space. The right main body portion 732F and the left main body portion 733F are stored in the storage case 790F so that they can be separated in the width direction W.

When the clip unit 1 is sandwiched between the right body portion 732F and the left body portion 733F, the stopper portion 731F restricts the clip unit 1 from moving downward in the height direction H until it is pushed in with a force equal to or greater than a predetermined value.

The sharp member 734F is a member provided near the opening 760 and has a sharp distal end. The sharp member 734F is a member that divides the connecting member 250.

The clip unit 1 is stored in each of the multiple clip storage areas 720. The connecting member 250 is arranged in the clip storage area 720 together with the clip unit 1. The connecting member 250 is attached to the clip unit 1.

Each of the multiple clip storage areas 720 is provided with an opening 760 into which the sheath 220 of the clip introduction device 200 can be inserted, and a lid 770 that closes the opening 760 in an independently openable manner. The opening 760 communicates with the sheath insertion area 740. When the opening 760 is closed by the lid 770, the clip storage area 720 is maintained in a sterile state.

The sheath 220 shown in FIG. 37 has been separated after the clip unit 1 has been used, and the connecting member 250 remains at the distal end.

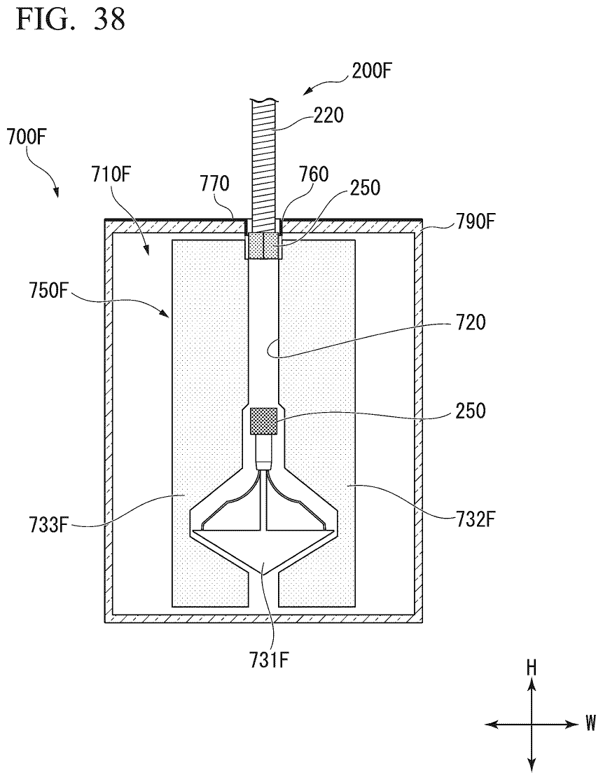

FIG. 38 shows the sheath 220 being inserted into the clip storage area 720.

The user presses the sheath 220 of the clip introduction device 200 against the seal 770 to break the seal 770 and insert the sheath 220 from the opening 760 into the clip storage area 720. At this time, the connecting member 250 comes into contact with the outer surface of the seal 770, which is a non-sterile area. That is, the connecting member 250 functions as a protective member that maintains the sterile state of the distal end of the sheath 220. The sterile state of the clip storage area 720 into which the sheath 220 has been inserted is released.

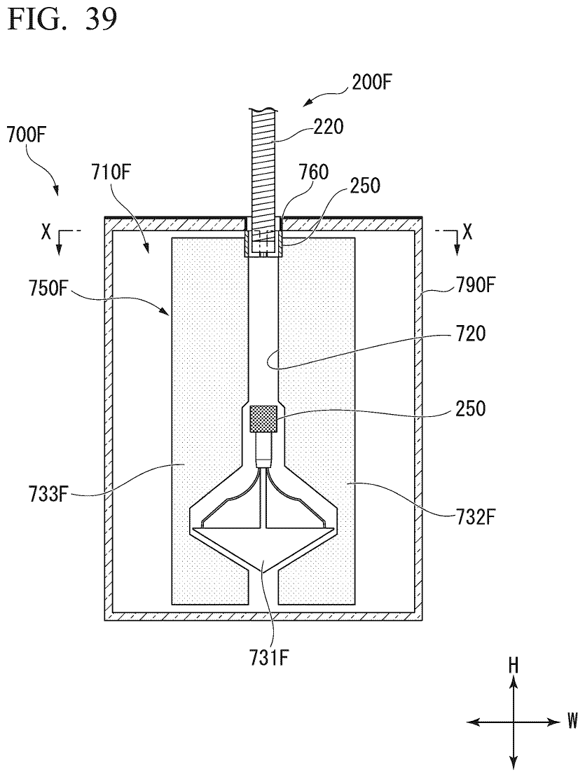

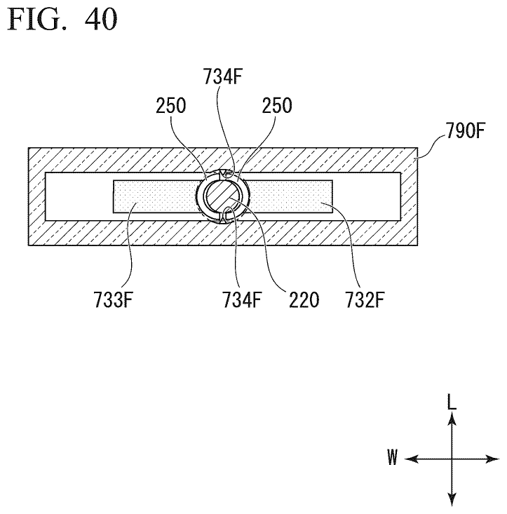

FIG. 39 shows the split connecting member 250. FIG. 40 is a cross-sectional view taken along line X-X shown in FIG. 39. The user further inserts the sheath 220 and brings the connecting member 250 into contact with the sharp member 734F. The connecting member 250 that has come into contact with the sharp member 734F splits and separates from the sheath 220.

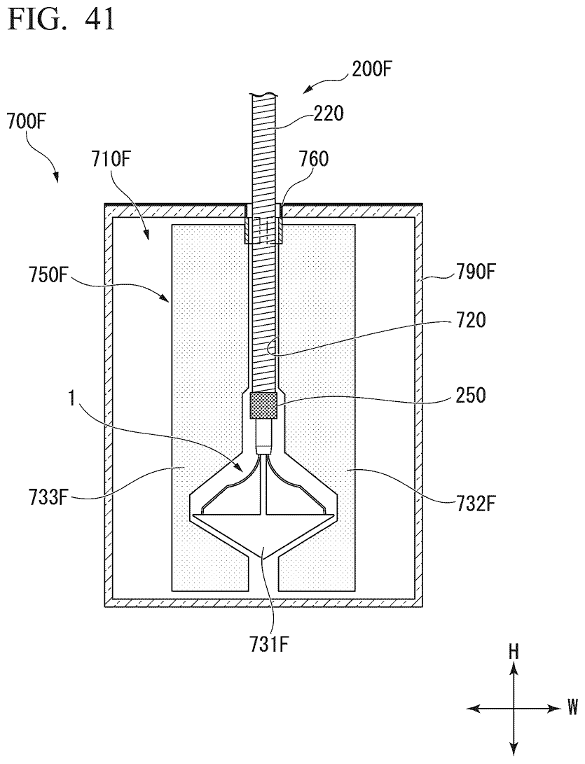

FIG. 41 shows the clip unit 1 after loading.

The user brings the distal chip 221 of the sheath 220 into contact with the connecting member 250 attached to the clip unit 1. The connecting member 250 connects the clip unit 1 and the distal chip (distal end portion) 221 of the sheath 220.

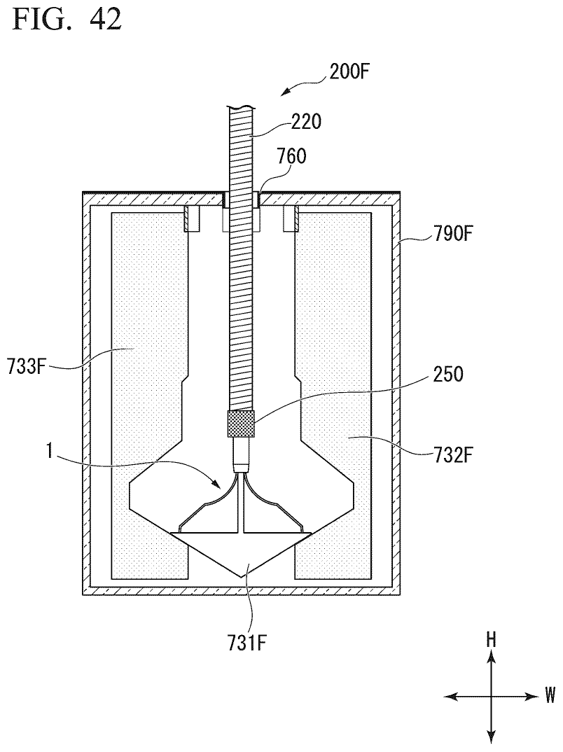

FIG. 42 shows the right main body portion 732F and the left main body portion 733F spaced apart in the width direction W.

The user pushes in the sheath 220 with a force equal to or greater than a predetermined value, thereby moving the stopper 731F downward in the height direction H. The stopper 731F that has moved downward separates the right main body portion 732F and the left main body portion 733F. As a result, a path through which the clip unit 1 passes is formed.

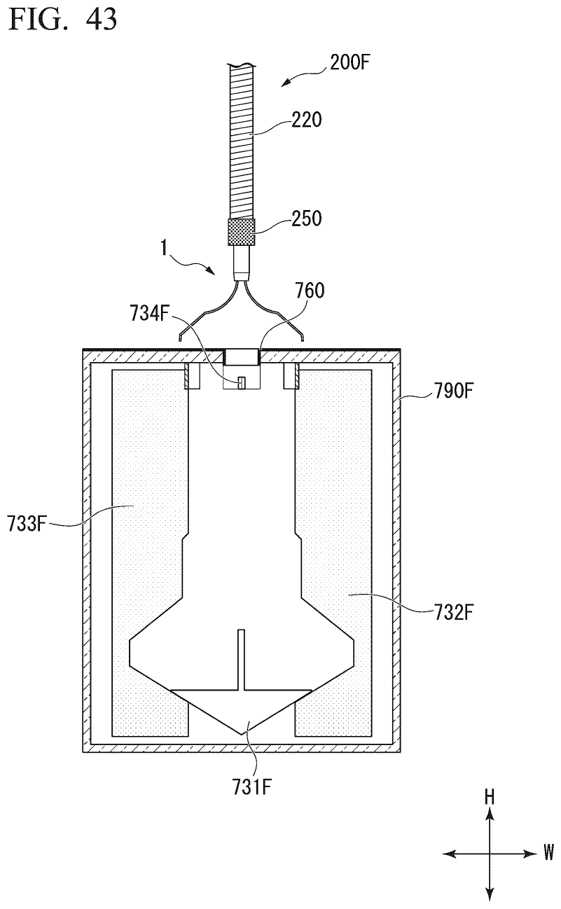

FIG. 43 shows the clip unit 1 removed from the storage case 790F.

The clip unit 1 loaded in the sheath 220 is removed from the storage case 790F by passing through a path formed between the separated right body portion 732F and left body portion 733F.

The connecting member 250 connecting the clip unit 1 and the sheath 220 functions as a protective member that protects the distal end of the sheath 220. When a new clip unit 1 is reloaded, the connecting member 250 comes into contact with the sharp member 734F, splits, and leaves the sheath 220.

According to the clip system 1000F of this embodiment, unused clip units 1 out of the multiple clip units 1 stored in the cartridge system 700B can be used for different cases. The clip system 1000F can keep the clip unit 1 and the sheath 220 clean when repeatedly reloading.

The fourth embodiment of the present disclosure has been described above in detail with reference to the drawings, but the specific configuration is not limited to this embodiment, and design modifications and the like that do not depart from the gist of the present disclosure are also included. In addition, the components shown in the above-mentioned embodiment and the modified examples shown below can be appropriately combined to form a configuration.

The present disclosure can be applied to cartridges that store clip units, etc.

What is claimed is:

1. A cartridge system, comprising:
a main body portion having a plurality of clip storage areas,
wherein each of the clip storage areas stores a clip, and
each of the clip storage areas is provided with an opening through which an applicator can be inserted and a lid for independently opening and closing the opening.

2. The cartridge system according to claim 1, wherein the clip storage area is maintained in a sterile state when the opening is closed by the lid.

3. The cartridge system according to claim 1, further comprising:
a base capable of supporting the main body portion with the opening disposed at a position where the applicator can be inserted.

4. The cartridge system according to claim 3, wherein the clip can be stored in the clip storage area when the main body portion and the base are separated.

5. The cartridge system according to claim 1, wherein the clip can be stored in the clip storage area before the lid is attached to the opening.

6. The cartridge system of claim 1, wherein the clip can be stored in the clip storage area when a part of the main body portion is removed from the main body portion.

7. The cartridge system of claim 1, wherein
the lid is a seal that closes the opening, and
the opening is opened when the applicator breaks through the seal and enters the clip storage area.

8. The cartridge system of claim 7, further comprising:
a mark that indicates a position where the applicator breaks through the seal.

9. The cartridge system of claim 1, wherein
the clip is stored in a case into which an applicator can be inserted, and
the case is housed in the clip storage area.

10. The cartridge system according to claim 1, further comprising a base,
wherein the main body portion has a first body portion and a second body portion,
the first body portion has a first clip storage area that is one of the plurality of clip storage areas,
the second body portion has a second clip storage area that is another of the plurality of clip storage areas,
the base is configured to support the first body portion and the second body portion independently and separably.

11. The cartridge system according to claim 10, wherein the base is configured to support the first body portion and the second body portion with the opening positioned at a position where the applicator can be inserted.

12. The cartridge system according to claim 1, wherein
the main body portion has a first main body portion and a second main body portion,
the first main body portion has a first clip storage area that is one of the plurality of clip storage areas,
the second main body portion has a second clip storage area that is another of the plurality of clip storage areas, and
the first main body portion and the second main body portion are separable.

13. The cartridge system according to claim 12, wherein
the first main body portion has a first base capable of supporting the first main body portion with the opening positioned at a position where the applicator can be inserted, and
the second main body portion has a second base capable of supporting the second main body portion with the opening positioned at a position where the applicator can be inserted.

14. The cartridge system according to claim 1, wherein
the main body portion has a first main body portion and a second main body portion,
the first main body portion has a first clip storage area that is one of the plurality of clip storage areas,
the second main body portion has a second clip storage area that is another of the plurality of clip storage areas, and
the first main body portion and the second main body portion are detachable.

15. The cartridge system according to claim 1, further comprising a storage case,
wherein the main body portion has a first main body portion and a second main body portion,
the first main body portion has a first clip storage area that is one of the plurality of clip storage areas,
the second main body portion has a second clip storage area that is another of the plurality of clip storage areas, and
the storage case stores the first main body portion and the second main body portion in a manner that allows them to be ejected in sequence.

16. The cartridge system according to claim 15, wherein, when the applicator is inserted into the first body portion that is disposed at an insertable position where the applicator can be inserted and the clip is loaded into the applicator, the first body portion is ejected from the storage case and the second body portion is disposed at the insertable position.

17. The cartridge system according to claim 16, wherein the second body portion is disposed inside the storage case when the first body portion is disposed at the insertable position.

18. The cartridge system according to claim 1, further comprising:
a protective member configured to protect a distal end of the applicator,
wherein, when the protective member opens the lid, the protective member separates from the applicator, and the applicator from which the protective member has been removed is inserted into the clip storage area.

19. The cartridge system of claim 18, further comprising:
a connecting member that detachably connects the clip and the applicator,
wherein the connecting member is disposed in the clip storage area together with the clip, and
the applicator inserted into the clip storage area is connected to the clip via the connecting member.

20. The cartridge system of claim 19, further comprising:
a connecting member that detachably connects the clip and the applicator,
wherein the connecting member is disposed in the clip storage area together with the clip, and
the applicator inserted into the clip storage area is connected to the clip via the connecting member.
